(19) United States
(12) Patent Application Publication
(10) Pub. No.: US 2022/0401452 A1
(43) Pub. Date: Dec. 22, 2022

(54) USE OF INHIBITORS OF ENHANCER OF ZESTE HOMOLOG 2

(71) Applicant: The Board Of Regents of The University of Texas System, Austin, TX (US)

(72) Inventors: Kexin Xu, San Antonio, TX (US); Chen-Hao Chen, Boston, MA (US); Xiaole Shirley Liu, Boston, MA (US); Myles Brown, Boston, MA (US)

(21) Appl. No.: 17/596,967

(22) PCT Filed: Jun. 26, 2020
(86) PCT No.: PCT/US2020/070201

§ 371 Date: Dec. 22, 2021

Related U.S. Application Data
(60) Provisional application No. 62/867,109, filed on Jun. 26, 2019.

(51) Int. Cl. A61K 31/5377; A61K 31/502; A61K 31/496; A61P 35/00; A61P 13/08; C12Q 1/6886

(52) U.S. Cl. CPC A61K 31/5377; A61K 31/502; A61K 31/496; A61P 35/00; A61P 13/08; C12Q 1/6886; C12Q 2600/158; C12Q 2600/106

(57) ABSTRACT

Described here are methods of assessing response of a patient to an EZH2 inhibitor and methods of treating certain cancers by administering therapeutically effective amounts of an EZH2 inhibitor and a PARP-1 inhibitor.

### CROSS REFERENCE TO RELATED APPLICATIONS

This application is a PCT application, and claims priority to, and the benefit of, U.S. Provisional Application No. 62/867,109, filed Jun. 26, 2019, titled “Use of Inhibitors of Enhancer of Zeste Homolog 2,” which is incorporated herein by reference in its entirety.

### GOVERNMENT SUPPORT

This invention was made with government support under grant numbers GM056211, HG004069, CA090381, CA163227, and CA178199 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Apr. 15, 2022, is named 205205_0031_1_(HSC-1583)_SL.txt and is 16,177 bytes in size.

### TECHNICAL FIELD

The present disclosure relates to methods of determining appropriateness of treating disease with inhibitors of Enhancer of Zeste Homolog 2 (EZH2). This disclosure also relates to methods of treating cancer using a combination of inhibitors of DNA damage repair proteins.

### BACKGROUND

EZH2 is a member of the Polycomb-Group (PcG) family of proteins that are involved in the regulation of the transcriptional state of genes by methylation of histone proteins. Originally identified as the catalytic subunit of the polycomb repressive complex 2 (PRC2), EZH2 methylates histone H3 at lysine 27 (H3K27) and leads to gene silencing. EZH2 is frequently upregulated in a broad spectrum of aggressive cancers and its overabundance is significantly associated with poor prognosis. Gain-of-function mutations at residues Y641, A677 or A687 within the catalytic domain of EZH2 have been identified in diffuse large B-cell lymphoma (DLBCL) and follicular lymphoma (FL). In view of these oncogenic features of EZH2, several selective inhibitors that block its enzymatic activity were developed. These compounds specifically inhibit EZH2-mediated methyl transfer reactions by competing with the methyl donor S-adenosylmethionine (SAM) for the binding pocket inside the catalytic domain. These prototype drugs abrogated the growth of non-Hodgkin lymphoma (NHL) cells that harbor EZH2 driver mutations, decreased global tri-methylation of H3K27 (H3K27me3), and re-activated genes that are repressed by PRC2 complex. But, it remains unclear whether the efficacy of EZH2 inhibitors will be limited to NHL harboring gain-of-function mutations or will be active as well on a range of solid tumors that rarely contain somatic mutations of EZH2.

Genotoxic stress, such as that induced by radiation or chemotherapy, predisposes cells to DNA damages and elicits diverse biological responses, including DNA repair, cell cycle arrest, and apoptosis. Deregulation of components critical for an appropriate DNA damage response (DDR) leads to genome instability, a hallmark of most cancers. Therefore, drugs that induce DNA damage or inhibit DDR, such as cisplatin and poly ADP ribose polymerase (PARP) inhibitors, are effective anticancer agents across a wide array of tumor types. EZH2 appears to play a pivotal role in determining how cancer cells respond to DNA damage. In one study, knockdown of EZH2 predominantly induced apoptosis in both p53-proficient and -deficient cancer cells. This was dependent on H3K27me3 (Histone 3 lysine 27 trimethylation)-mediated epigenetic silencing of F-box only protein 32 (FBXO32), which is required for p21 protein degradation. In another report, depletion of EZH2 rapidly prompted a senescence-related DDR via activation of ATM (ataxia-telangiectasia mutated kinase)-p53-p21 pathway. No changes in H3K27me3 pattern or overall level were observed during the process. In addition, EZH2 was found to be essential for cell proliferation following escape from senescence, but again changes in H3K27me3 were not involved in this phenotypic adaptation. It was recently reported that EZH2 directly binds to the internal ribosome entry site (IRES) on the mRNAs of both wild-type and mutated p53, leading to enhanced translation of p53 protein, especially the mutant one, and subsequently promoted cancer growth and metastasis. However, this EZH2-RNA interaction seems to be independent of EZH2's methyltransferase activity. These studies present a diverse and complex picture of EZH2 functions in regulation of DDR. Additionally, the role of H3K27me3 in the EZH2-mediated DDR is unclear. It is challenging to evaluate if a patient would be impacted by treatment with inhibitors of EZH2. Moreover, the influence of the components involved in the DDR over the oncogenic functions of EZH2 has not been elucidated. Similarly, it is challenging to assess which combination of inhibitors of DNA damage repair proteins will be effective for a patient.

### SUMMARY

Disclosed herein are compositions and methods addressing the shortcomings of the art, and may provide any number of additional or alternative advantages. Provided here is a method of assessment of sensitivity of cancer cells to EZH2 inhibitors based on a gene expression signature of certain DNA damage repair proteins. These genes are targeted by EZH2 inhibitors and underlie the tumor-suppressive effects of these EZH2 inhibitors. In an embodiment, the EZH2 inhibitors are selected to sensitize cancers that overexpress EZH2-regulated DNA damage repair genes to genotoxic agents.

Embodiments include methods of treating cancer in a subject. One such method includes administering therapeutically effective amounts of an EZH2 inhibitor and a PARP-1 inhibitor. In an embodiment, the cancer is prostate cancer. In an embodiment, the PARP-1 inhibitor is one or more of olaparib, rucaparib, niraparib (MK4827), talazoparib (BMN673), veliparib (ABT-888), iniparib, pamiparib, 3-Aminobenzamide (INO-1001), E7016 (GPI21016), CEP-8963, and CEP-9722. In an embodiment, the EZH2 inhibitor is one or more of tazemetostat, 3-deazaneplanocin A (DZNep), EPZ005687, EI1, GSK126, EPZ-6438, GSK343, GSK503, CPI-1205, Constellation Compound 3, +OR-52, and +UNC1999.

Embodiments include methods of assessing response of cancer cells from a patient to an EZH2 inhibitor. One such method includes assessing response of cancer cells from a patient to multiple EZH2 inhibitors. Another such method includes obtaining a biological sample containing cancer cells from a patient and evaluating the expression of a group of DNA damage repair genes to measure sensitivity of the cancer cells to EZH2 inhibitors, either as monotherapy or in combination with DNA damaging agents.

In an embodiment, this method includes comparing expression levels of a plurality of biomarkers in a first biological specimen from a patient to expression levels of the plurality of biomarkers in a second biological specimen from the patient, wherein the first biological specimen contains cancer cells and the second biological specimen contains normal control cells of the patient and a change in the expression levels of the plurality of biomarkers indicates a response to one or more EZH2 inhibitors.

Embodiments include methods of assaying sensitivity of cancer cells of a patient to an EZH2 inhibitor or assessing therapeutic efficacy of an EZH2 inhibitor for a particular patient. One such method includes comparing expression levels of a plurality of biomarkers in a biological specimen from a patient to reference expression levels of the plurality of biomarker genes from normal cells. A change in the expression levels of the plurality of biomarkers indicates sensitivity of cancer cells of a patient to an EZH2 inhibitor or the therapeutic efficacy of the EZH2 inhibitor. In an embodiment, the biological specimen includes prostate cancer cells. In an embodiment, the plurality of biomarkers includes genes or gene expression products or fragments thereof from one or more of the base excision repair, mismatch repair, Fanconi anemia pathway, homologous recombination, or non-homologous end-joining pathways, as described in the Kyoto Encyclopedia of Genes and Genomes (KEGG). In an embodiment, the plurality of biomarkers includes genes or gene expression products or fragments thereof from the following: Cell Division Cycle-Associated Protein 3 (CDCA3), CDC28 Protein Kinase Regulatory Subunit 2 (CKS2), MutY DNA Glycosylase (MUTYH), DNA Polymerase Epsilon 3 (POLE3), Transforming Acidic Coiled-Coil Containing Protein 3 (TACC3), Replication Factor C Subunit 4 (RFC4), Replication Factor C Subunit 2 (RFC2), REV3 Like, DNA Directed Polymerase Zeta Catalytic Subunit (REV3L), DNA Polymerase Delta 4, Accessory Subunit (POLD4), MutL Homolog 1 (MLH1), FA Complementation Group G (FANCG), FA Complementation Group F (FANCF), FA Complementation Group L (FANCL), Breast Cancer Type 1 Susceptibility Protein (BRCA1), DNA Polymerase Epsilon 2, Accessory Subunit (POLE2), BRCA1 Interacting Protein C-Terminal Helicase 1 (BRIP1), Protein Kinase, DNA-Activated, Catalytic Subunit (PRKDC), DNA Polymerase Iota (POLI), Nei Like DNA Glycosylase 1 (NEIL1), Poly(ADP-Ribose) Polymerase Family Member 3 (PARP3), DNA Polymerase Mu (POLM), DNA Ligase 1 (LIG1), BRCA1 Associated RING Domain 1 (BARD1), DNA Cross-Link Repair 1C (DCLRE1C), Ubiquitin Conjugating Enzyme E2 T (UBE2T), N-Methylpurine DNA Glycosylase (MPG), Flap Structure-Specific Endonuclease 1 (FEN1), RB Binding Protein 8 Endonuclease (RBBP8), Replication Factor C Subunit 3 (RFC3), BRCA1/BRCA2-Containing Complex Subunit 3 (BRCC3), FA Complementation Group D2 (FANCD2), Replication Factor C Subunit 5 (RFC5), DNA Polymerase Epsilon, Catalytic Subunit (POLE), DNA Polymerase Beta (POLB), Hes Family BHLH Transcription Factor 1 (HES1), RAD54 Like (RAD54L), DNA Nucleotidylexotransferase (DNTT), MutS Homolog 2 (MSH2), DNA Polymerase Delta 1 Catalytic Subunit (POLD1), BRCA2 DNA Repair Associated (BRCA2), DNA Polymerase Epsilon 4, Accessory Subunit (POLE4), Poly(ADP-Ribose) Polymerase 1 (PARP1), MutL Homolog 3 (MLH3), Nei Like DNA Glycosylase 3 (NEIL3), BLM RecQ Like Helicase (BLM), Replication Protein A2 (RPA2), Replication Protein A3 (RPA3), RAD50 Double Strand Break Repair Protein (RAD50), Nth Like DNA Glycosylase 1 (NTHL1), DNA Ligase 4 (LIG4), DNA Polymerase Eta (POLH), X-Ray Repair Cross Complementing 4 (XRCC4), Nei Like DNA Glycosylase 2 (NEIL2), RecQ Mediated Genome Instability 2 (RMI2), FA Complementation Group I (FANCI), Uracil DNA Glycosylase (UNG), MutS Homolog 6 (MSH6), Exonuclease 1 (EXO1), ATM Serine/Threonine Kinase (ATM), Nibrin (NBN), RecQ Mediated Genome Instability 1 (RMI1), High Mobility Group Box 1 (HMGB1), and RAD51 Paralog C (RAD51C). In an embodiment, the plurality of biomarkers includes genes or gene expression products or fragments thereof from three or more of CDCA3, CKS2, MUTYH, POLE3, TACC3, RFC4, RFC2, REV3L, POLD4, MLH1, FANCG, FANCF, FANCL, BRCA1, POLE2, BRIP1, PRKDC, POLI, NEIL1, PARP3, POLM, LIG1, BARD1, DCLRE1C, UBE2T, MPG, FEN1, RBBP8, RFC3, BRCC3, FANCD2, RFC5, POLE, POLB, HES1, RAD54L, DNTT, MSH2, POLD1, BRCA2, POLE4, PARP1, MLH3, NEIL3, BLM, RPA2, RPA3, RAD50, NTHL1, LIG4, POLH, XRCC4, NEIL2, RMI2, FANCI, UNG, MSH6, EXO1, ATM, NBN, RMI1, HMGB1, and RAD51C. Significant change is measured by determining the mean expression level of the biomarkers from the first biological specimen from a patient and comparing them to the reference expression levels of the plurality of biomarker genes from normal cells. A significant increase of mean expression value is defined as higher than mean plus one standard deviation of expression distribution between the specimen and normal cells. The exact threshold and units would be dependent on the detection platform used.

Embodiments include a set of reagents to measure the levels of the plurality of biomarkers in a specimen, wherein the biomarkers are one or more of CDCA3, CKS2, MUTYH, POLE3, TACC3, RFC4, RFC2, REV3L, POLD4, MLH1, FANCG, FANCF, FANCL, BRCA1, POLE2, BRIP1, PRKDC, POLI, NEIL1, PARP3, POLM, LIG1, BARD1, DCLRE1C, UBE2T, MPG, FEN1, RBBP8, RFC3, BRCC3, FANCD2, RFC5, POLE, POLB, HES1, RAD54L, DNTT, MSH2, POLD1, BRCA2, POLE4, PARP1, MLH3, NEIL3, BLM, RPA2, RPA3, RAD50, NTHL1, LIG4, POLH, XRCC4, NEIL2, RMI2, FANCI, UNG, MSH6, EXO1, ATM, NBN, RMI1, HMGB1, RAD51C, or measurable fragments thereof. In an embodiment, the set of reagents to measure the levels of the plurality of biomarkers in a specimen, wherein the biomarkers are one or more of CDCA3, CKS2, MUTYH, POLE3, TACC3, or measurable fragments thereof. The biomarkers can be used in various combinations for diagnosis. The disclosure provides biomarkers, methods, devices, reagents, systems, and kits for assessing response of cancer cells from a patient to an EZH2 inhibitor or assessing therapeutic efficacy of an EZH2 inhibitor for treating a patient afflicted with cancer.

Embodiments include a method for sensitizing cancer cells in a subject in need of chemotherapy by administering to the subject a therapeutically effective amount of an Enhancer of Zeste Homolog 2 (EZH2) inhibitor before administration of a poly (ADP-ribose) polymerase 1 inhibitor. These cancer cells in the subject are characterized by elevated levels of expression of one or more of Cell Division Cycle-Associated Protein 3, CDC28 Protein Kinase Regulatory Subunit 2, MutY DNA Glycosylase, DNA Polymerase Epsilon 3, and Transforming Acidic Coiled-Coil Containing Protein 3 as compared to normal cells.

Numerous other aspects, features and benefits of the present disclosure may be made apparent from the following detailed description taken together with the figures. The compositions can include pharmaceutical compositions described herein along with other components, or ingredients depending on desired prevention and treatment goals. It should be further understood that both the foregoing general description and the following detailed description are exemplary and explanatory and are intended to provide further explanation of the various inventions as claimed.

### BRIEF DESCRIPTION OF THE DRAWINGS

Embodiments will be readily understood by the following detailed description in conjunction with the accompanying drawings. Embodiments are illustrated by way of example and not by way of limitation in the figures of the accompanying drawings.

FIG. 1A is a diagrammatic representation of the work flow of CRISPR knockout screens in an androgen-dependent prostate cancer cell line (LNCaP) cell line and an LNCaP-derived, castration-resistant counterpart—abl cell line. FIG. 1B is a graphical representation of the difference of gene essentialities, represented by beta (β) scores, between abl and LNCaP [β(abl)-β(LNCaP)] for each individual gene. Positions of representative genes were indicated by red dots. FIG. 1C is a graphical representation of the IC50 for two EZH2 inhibitors (GSK126 represented by red bars; EPZ-6438 represented by blue bars) in a panel of prostate normal and cancer cell lines after 6 days of treatment. FIG. 1D is a graphical representation of the effects of EZH2 inhibitors (left panel for GSK126, and right panel for EPZ-6438) on cell growth over time in abl cells with indicated concentrations of the compounds. FIG. 1E is a graphical representation of the effects of EZH2 inhibitors (left panel for GSK126, and right panel for EPZ-6438) on cell growth over time in LNCaP cells with indicated concentrations of the compounds. FIG. 1F is a growth curve of xenograft tumors in castrated nude mice injected with CWR22Rv1 cells receiving vehicle (Veh) or GSK126, at the indicated doses. FIG. 1G is a growth curve of xenograft tumors in castrated nude mice injected with CWR22Rv1 cells receiving vehicle (Veh) or EPZ-6438, at the indicated doses.

FIG. 2A is a heat map of differential gene expression patterns in abl cells being treated with vehicle (DMSO), 5 μM GSK126 (GSK) or 5 μM EPZ-6438 (EPZ) for 72 hrs. FIG. 2B is an illustration of overrepresented functional annotations of genes that were significantly downregulated upon the treatment with EZH2 inhibitors in abl cells from Gene Set Enrichment Analysis. Blue bars represent the percentage of genes in each specific functional category. The red line represents values of the false discovery rate (FDR) for the particular GO term. FIG. 2C is a heat map of differential genes induced by EZH2 inhibitors (GSK, GSK126; EPZ, EPZ-6438) comparing to vehicle control (GSK vs. DMSO and EPZ vs. DMSO) or EZH2 knockdown comparing to control siRNA (siEZH2 vs. siCtrl) in abl cells. FIG. 2D is a heat map of quantitative real-time RT-qPCR results showing changes in mRNA levels of selected gene in three prostate cancer cell lines (C4-2B, LAPC4-CR and LNCaP-AI). Cells were treated with vehicle (DMSO), 5 μM GSK126 (GSK) or 5 μM EPZ-6438 (EPZ) for 72 hrs. Top panels, EZH2-activated genes; bottom panels, EZH2-repressed genes. FIGS. 2E-2J are graphical representation of the mRNA expression of the following genes in xenograft tumor tissues from control mice (Veh) or mice treated with GSK126 (GSK) or EPZ-6438 (EPZ). Numbers, triplicates of samples from control or treatment group: KIAA0101 (FIG. 2E), CDCA3 (FIG. 2H), BIRC5 (FIG. 2F), POLE3 (FIG. 2I), POLE (FIG. 2G), and MUTYH (FIG. 2J). FIG. 2K is a graphical representation of the protein levels of selected genes in xenograft tumor tissues from control mice (Veh) or mice treated with GSK126 (GSK) or EPZ-6438 (EPZ). Numbers, triplicates of samples from control or treatment group.

FIG. 3A is a photographic image of Western blots of H3K27 methylation levels in abl cells that were replaced with the control (Vector), the wild type (WT) or EZH2 mutants bearing different point mutations (Y111D or Y661D). Cells were treated with DMSO or EZH2 inhibitors (GSK, GSK126; EPZ, EPZ-6438) for 72 hrs. FIG. 3B is a graphical representation of the IC50 values for two EZH2 inhibitors in abl cells expressing the control (Vector), the wild-type EZH2 (WT) or EZH2 mutants bearing different point mutations (Y111D or Y661D). Cells were incubated with EZH2 inhibitors (GSK126, red bars; EPZ-6438, blue bars) for 6 days and then collected for direct counting after trypan blue staining. FIG. 3C is a graphical representation of effects of EZH2 inhibitors (GSK referring to GSK126, and EPZ referring to EPZ-6438) on the growth of abl cells expressing the control (Vector) construct at indicated time points. FIG. 3D is a graphical representation of effects of EZH2 inhibitors (GSK referring to GSK126, and EPZ referring to EPZ-6438) on the growth of abl cells expressing the wild-type EZH2 (WT) at indicated time points. FIG. 3E is a graphical representation of effects of EZH2 inhibitors (GSK referring to GSK126, and EPZ referring to EPZ-6438) on the growth of abl cells expressing the EZH2 mutant—Y111D—at indicated time points. FIG. 3F is a graphical representation of effects of EZH2 inhibitors (GSK referring to GSK126, and EPZ referring to EPZ-6438) on the growth of abl cells expressing the EZH2 mutant—Y661D—at indicated time points. FIG. 3G is a diagrammatic representation of the expression of EZH2-activated genes as detected by RT-qPCR in abl cells that were replaced with the control (Vector), the wild-type EZH2 (WT) or the mutants in the presence of vehicle (DMSO) or 5 μM EZH2 inhibitors (GSK, GSK126; EPZ, EPZ-6438) for 3 days.

FIGS. 4A and 4B are photographic images of the H3K27 methylation levels in prostate cell lines with the treatment of vehicle (DMSO), GSK126 (GSK) or EPZ-6438 (EPZ) at specified doses (1 or 5 μM final concentration). FIG. 4C is a set of scatter plots of H3K27me3 peak signals, after being normalized to the Drosophila reference epigenome, in abl cells (left panel) and DU145 cells (right panel) under control condition (x-axis) or after the treatment with EZH2 inhibitors (y-axis). FIG. 4D is a graphical representation of the EZH2 inhibitor-induced changes in H3K27me3 levels between abl and DU145 cells after SPIKE-IN normalization (GSK referring to GSK126, and EPZ referring to EPZ-6438; F.C., fold change). FIG. 4E is a graphical representation of the direct ChIP-qPCR results of H3K27me3 at selected chromatin regions after abl cells were treated with control (DMSO) or EZH2 inhibitors (GSK126 or EPZ-6438) for indicated number of days. KIAA0066 and PPIA are negative controls. FIG. 4F is a photographic image of an immunoblot of H3K27me3 protein levels in the corresponding ChIP samples (GSK referring to GSK126, and EPZ referring to EPZ-6438). FIG. 4G is a graphical representation of the SPIKE-IN normalized signals of H3K27me3 peaks around genes that were upregulated (EZH2-repressed), downregulated (EZH2-activated) or showed no differences (non-differential) upon the treatment with EZH2 inhibitors. Intensities of the histone mark under either control (DMSO) or treatment condition (GSK referring to GSK126, and EPZ referring to EPZ-6438) were plotted.

FIG. 5A is a diagrammatic representation of the work flow of CRISPR-Cas9 knockout screening, which targets 6,000 cancer-related genes in LNCaP and abl cells in the presence of vehicle or GSK126 (EZH2 inhibitor) for four weeks. FIG. 5B is a graphical representation of the distribution of delta beta (Δβ) scores, defined as beta scores under treatment (+GSK126) condition minus beta scores under control (+Veh.) condition [β(GSK)-β(DMSO)]. Representative DNA repair genes were indicated by red dots. FIG. 5C is a graphical representation of the gene set enrichment analysis of genes with positive delta beta scores (Δβ) in CRISPR-Cas9 knockout screening in abl cells. Blue bars, percentage of genes in each specific functional category; red line, values of the false discovery rate (FDR) for the particular gene ontology term. FIG. 5D is a heat map showing differential expression (log-transformed) of genes involved in the specified DNA damage repair pathways upon treatment with EZH2 inhibitors. Transcript levels of these genes in abl and DU145 cells were compared between treatment groups and control group (EPZ vs. DMSO and GSK vs. DMSO). FIG. 5E is a graphical representation of the percentages of genes containing two types of EZH2 chromatin binding (EZH2_H3K27me3 low, EZH2 binding sites with low or no H3K27me3 signals; EZH2_H3K27me3 high, EZH2 binding sites with high H3K27me3 signals) within 1 kb around their transcriptional start sites. Blue bars, genes involved in DNA damage repair pathways; rose red bars, genes not involved in DNA damage repair.

FIG. 5F is a graphical representation of the heat map showing expression of DNA repair genes in primary or metastatic prostate cancer. Genes were categorized into different DNA damage repair pathways according to their functions. FIG. 5G is a graphical representation of the expression correlation between EZH2 and DNA repair genes in a prostate cancer cohort. Each dot represents a clinical sample, with primary prostate cancer (PCa) samples colored in blue while the metastatic ones colored in yellow.

FIG. 6A is a graphical representation of the alkaline comet assays in abl cells. Prostate cancer cells were pretreated with vehicle (DMSO) or 5 μM GSK126 (GSK) for 7 days, and then exposed to increasing dosages of ionizing radiation (IR) followed by recovery for 4 hrs. FIG. 6B is a set of photographic images of each IR dosage corresponding to the dosages in FIG. 6A. FIG. 6C is a graphical representation of the alkaline comet assays in LNCaP cells. Prostate cancer cells were pretreated with vehicle (DMSO) or 5 μM GSK126 (GSK) for 7 days, and then exposed to increasing dosages of ionizing radiation (IR) followed by recovery for 4 hrs. FIG. 6D is a set of photographic images of each IR dosage corresponding to the dosages in FIG. 6C. FIG. 6E is a graphical representation of the alkaline comet assays in DU145 cells. Prostate cancer cells were pretreated with vehicle (DMSO) or 5 μM GSK126 (GSK) for 7 days, and then exposed to increasing dosages of ionizing radiation (IR) followed by recovery for 4 hrs. FIG. 6F is a set of photographic images of each IR dosage corresponding to the dosages in FIG. 6D. FIG. 6G is a graphical representation of the combination therapy of EZH2 inhibitor and olaparib to abrogate the androgen-independent growth of abl cells. Cells were treated with vehicle (DMSO), 0.5 μM GSK126 (GSK) alone, 1 μM Olaparib alone, or both drugs (GSK+Olaparib) for indicated days, and cell proliferation were determined using Cell Counting Kit-8.

FIG. 7A is a graphical representation of the expression correlation between EZH2 and DNA damage repair genes in cancer cells from CCLE gene expression data. Each dot represents one cell line. FIG. 7B is a box plot showing the sensitivity of CTRP cell lines to EZH2 inhibitor (BRD, BRD-K62801835-001-01-0). Cells were grouped based on their mean expression of DNA damage repair genes, and then sensitivities to BRD in the top 25% cells with the highest expression were compared with those in bottom 25% cells with the lowest expression. FIG. 7D is a box plot showing the sensitivity of CTRP cell lines to EZH2 inhibitor (BRD, BRD-K62801835-001-01-0). Cells were grouped based on their mean expression of EZH2-repressed genes, and then sensitivities to BRD in the top 25% cells with the highest expression were compared with those in bottom 25% cells with the lowest expression. FIG. 7C is a box plot illustrating the correlation between expression of DNA damage repair genes and dependency of EZH2. EZH2 dependency scores (CERES scores) of all the cell lines were retrieved from the DepMap CRISPR-Cas9 knockout screening data. FIG. 7E is a box plot showing the sensitivity to EZH2 inhibitor (BRD, BRD-K62801835-001-01-0) in three different groups of cancer cells based on the statuses of EZH2 mutations. All the lymphoma and leukemia cells were first separated from solid tumors, with further classification according to the presence of EZH2 somatic mutations. GOF, gain-of-function mutations at residues Y641, A677 or A687; LOF, loss-of-function mutations; None, no mutations. FIG. 7F is box plot illustrating the correlation between the expression of DNA damage repair genes and sensitivity of cancer cells to EZH2 inhibitor (BRD, BRD-K62801835-001-01-0). Cells containing EZH2 somatic mutations were excluded from the analysis.

FIG. 8A is a graph of gene set enrichment analysis (GSEA) result showing positive sgRNAs that are known to regulate cell proliferation were indeed negatively selected in both LNCaP and abl cells. FIG. 8B is a graph of distributions of beta scores from the CRISPR-Cas9 knockout screening in LNCaP (x-axis) and abl (y-axis) cells. Position of EZH2 was indicated by red dots. Numbers in the parentheses, beta scores in LNCaP (the first values) and abl (the second values).

FIG. 9A is the growth curve of DU145 cells over time with indicated concentrations of EZH2 inhibitors (GSK126, left panel and EPZ-6438, right panel). FIG. 9B is the box plot showing cell cycle analysis of abl cells with the treatment of vehicle (DMSO), 5 μM GSK126 (GSK) or 5 μM EPZ-6438 (EPZ) over indicated days. FIG. 9C is the box plot showing prostate cancer cells (C4-2B, LAPC4-CR and DU145) after being treated with vehicle (DMSO), 5 μM GSK126 (GSK) or 5 μM EPZ-6438 (EPZ) for 3 days, and then subjected to propidium iodide staining followed by flow cytometry analysis.

FIG. 10A is the heat map of differential genes in DU145 cells, which were treated with vehicle (DMSO), 5 μM GSK126 (GSK) or 5 μM EPZ-6438 (EPZ) for 72 hrs. FIGS. 10B-10G are bar plots demonstrating the effect of EZH2 knockdown in DU145 cells on the expression of ETV4 (FIG. 10B), FOXC1 (FIG. 10C), GATA6 (FIG. 10D), HOXD8 (FIG. 10E), NFB2 (FIG. 10F), and RUNX3 (FIG. 10G).

FIGS. 11A-11J are bar plots demonstrating the effect of EZH2 inhibitors (GSK126 or EPZ-6438) on expression of the following genes in abl cells at different concentrations for 72 hrs: CDCA3 (FIG. 11A), CKS2 (FIG. 11B), KCNMB4 (FIG. 11C), MAT1A (FIG. 11D), MUTYH (FIG. 11E), POLE3 (FIG. 11F), SHH (FIG. 11G), TNF SF9 (FIG. 1111), TACC3 (FIG. 11I), and VIM (FIG. 11J). FIGS. 11K-11T are bar plots demonstrating the effect of EZH2 inhibitors (GSK126 or EPZ-6438) on expression of the following genes in abl cells at 5 μM for indicated days: CKS2 (FIG. 11K), CDCA3 (FIG. 11L), KCNMB4 (FIG. 11M), MAT1A (FIG. 11N), MUTYH (FIG. 11O), POLE3 (FIG. 11P), TACC3 (FIG. 11Q), SHH (FIG. 11R), TNFSF9 (FIG. 11S), and VIM (FIG. 11T).

FIGS. 12A-12D are graphical representations of abl cells expressing the control (Vector) (FIG. 12A), the wild-type EZH2 (WT) (FIG. 12B), EZH2 mutant (Y111D) (FIG. 12C), and EZH2 mutant (Y661D) (FIG. 12D) when incubated with indicated concentrations of GSK126 for 6 days. FIGS. 12E-12H are graphical representations of abl cells expressing the control (Vector) (FIG. 12E), the wild-type EZH2 (WT) (FIG. 12F), EZH2 mutant (Y111D) (FIG. 12G), and EZH2 mutant (Y661D) (FIG. 12H) when incubated with indicated concentrations of EPZ-6438 (B) for 6 days. FIGS. 12I-12L are graphical representations of expression of the following EZH2-repressed genes upon EZH2 inhibitor treatment (GSK126 or EPZ-6438) in abl cells expressing the control (Vector), the wild-type EZH2 (WT), EZH2 mutant (Y111D), and EZH2 mutant (Y661D): MATA1A (FIG. 12I), SHH (FIG. 12J), VIM (FIG. 12K), and TNFSF9 (FIG. 12L).

FIG. 13A is a representation of the intensity of each H3K27me3 peak as compared between control condition (DMSO, x-axis) and treatment condition (EZH2 inhibitors, y-axis) in abl cells, using reads per million methods. FIG. 13B is a representation of the H3K27me3 peak enrichment under the conditions of vehicle (DMSO), 5 μM GSK126 (GSK) or 5 μM EPZ-6438 (EPZ) as normalized using canonical method and then compared between abl and DU145 cells. FIG. 13C is a graphical representation of direct ChIP-qPCR of H3K27me3 as performed and detected at selected chromatin regions in DU145 cells. FIG. 13D is a photographic image of the immunoblot of H3K27me3 protein levels in the corresponding ChIP samples.

FIG. 14A is a graphical representation of distribution of the beta scores from the CRISPR-Cas9 knockout screening in LNCaP cells under control condition (+Veh., x-axis) and treatment condition (+GSK126, y-axis). FIG. 14B is a graphical representation of the difference of gene essentialities, represented by beta (β) scores, between vehicle treatment (DMSO) and EZH2 inhibitor treatment (GSK) for each individual gene. Positions of representative genes were indicated by red dots. FIG. 14C is a graph showing functional annotations that were enriched in genes with positive delta beta scores from CRISPR-Cas9 knockout screening in LNCaP cells. FIG. 14D is a heat map showing expression of DNA damage repair genes in an independent prostate cancer cohort containing primary or metastatic tumors. Genes were categorized into different DNA repair pathways according to their functions. FIG. 14E is a dot plot showing expression correlation between EZH2 and DNA damage repair genes in an independent prostate cancer cohort. Each dot represents a clinical case, and all the primary prostate cancer (PCa) were colored in blue, while the metastatic ones were colored in yellow. FIG. 14F is a heat map demonstrating the transcript levels of EZH2-activated genes that are functionally involved in either base excision repair or mismatch repair pathway among LNCaP, abl, and DU145.

FIG. 15A is a graphical representation of the alkaline comet assay results in abl cells after pretreatment with vehicle (DMSO) or 5 μM GSK126 (GSK) for 7 days and then being exposed to 5 gray (Gy) ionizing radiation (IR) followed by recovery at indicated time points (Quantification of the results using “Olive Tail Moment” parameter). FIG. 15B is a set of representative images of each time point corresponding the sampling in FIG. 15A. FIG. 15C is a graphical representation of the alkaline comet assay results in LNCaP cells after pretreatment with vehicle (DMSO) or 5 μM GSK126 (GSK) for 7 days and then being exposed to 5 gray (Gy) ionizing radiation (IR) followed by recovery at indicated time points (Quantification of the results using “Olive Tail Moment” parameter). FIG. 15D is a set of representative images of each time point corresponding the sampling in FIG. 15C. FIG. 15E is a graphical representation of the alkaline comet assay results in DU145 cells after pretreatment with vehicle (DMSO) or 5 μM GSK126 (GSK) for 7 days and then being exposed to 5 gray (Gy) ionizing radiation (IR) followed by recovery at indicated time points (Quantification of the results using “Olive Tail Moment” parameter). FIG. 15F is a set of representative images of each time point corresponding the sampling in FIG. 15E. FIG. 15G is box plot showing the olive tail moment values in abl, LNCaP and DU145 cell after being treated with 5 gray (Gy) ionizing radiation (IR) followed by recovery at indicated time points.

FIG. 16A is a graph showing expression correlation between EZH2 and DNA damage repair genes within one particular type of cancers from TCGA data. Each dot represents one cancer type; color scale, correlation coefficient of all samples within individual cancer type. FIG. 16B is a scatter plot illustrating the expression correlation between EZH2 and EZH2-repressed genes from CCLE data. FIG. 16C is a dot plot representing expression levels of EZH2 (red dots) and DNA damage repair genes (blue dots) in each specified cancer type in TCGA data.

### DETAILED DESCRIPTION

Reference will now be made to the exemplary embodiments illustrated in the drawings, and specific language will be used here to describe the same. It will nevertheless be understood that no limitation of the scope of the invention is thereby intended. Alterations and further modifications of the inventive features illustrated here, and additional applications of the principles of the inventions as illustrated here, which would occur to one skilled in the relevant art and having possession of this disclosure, are to be considered within the scope of the invention.

As used herein, unless otherwise noted, the terms “treating”, “treatment” and the like, shall include the management and care of a subject or patient (preferably mammal, more preferably human) for the purpose of combating a disease, condition, or disorder and includes the administration of a compound of the present invention to prevent the onset of the symptoms or complications, alleviate the symptoms or complications, or eliminate the disease, condition, or disorder. The term “subject” as used herein, refers to an animal, preferably a mammal, most preferably a human, who is the object of treatment, observation, or experiment. Preferably, the subject has experienced and/or exhibited at least one symptom of the disease or disorder to be treated and/or prevented. The term “therapeutically effective amount” as used herein, means that amount of active compound or pharmaceutical agent that elicits the biological or medicinal response in a tissue system of a subject, which is being sought by a researcher, veterinarian, medical doctor or other clinician, which includes treating the disease or disorder in the subject. Pharmaceutically acceptable derivative of an EZH2 inhibitor includes any pharmaceutically acceptable salt, solvate, hydrate or prodrug of such EZH2 inhibitor. Pharmaceutically acceptable derivative of a PARP inhibitor includes any pharmaceutically acceptable salt, solvate, hydrate or prodrug of such PARP inhibitor.

Disclosed here are agents that inhibit the activity of EZH2 to promote anticancer effects. These agents are used for treating cancer in a subject. EZH2 inhibitors are used in clinical trials for lymphoma or leukemia, especially those with EZH2 gain-of-function mutants. Embodiments disclosed here include EZH2 inhibitors for use in the treatment of solid tumors. Embodiments include methods of chemotherapy involving EZH2 inhibitors and PARP inhibitor in cancers with high DNA damage repair gene expression. In certain embodiments, EZH2 inhibitors are utilized to enhance sensitivity of cancer cells to DNA repair targeted therapies, such as ionizing radiation and olaparib. In certain embodiments, EZH2 inhibitors are utilized as part of a combination therapy with radiotherapy or PARP inhibitor, such as olaparib, to treat a wide range of advanced cancer. Patients can be treated with pharmaceutical compositions containing a therapeutically effective amount of an EZH2 inhibitor and a therapeutically effective amount of a PARP inhibitor. In an embodiment, the cancer is prostate cancer. In an embodiment, EZH2 inhibitors (EZH2i) abrogate the growth of prostate cancer cells, especially those that have become castration resistant. The PARP-1 inhibitor can be one or more of olaparib, rucaparib, niraparib (MK4827), talazoparib (BMN673), veliparib (ABT-888), iniparib, pamiparib, 3-Aminobenzamide (INO-1001), E7016 (GPI21016), CEP-8963, and CEP-9722. The EZH2 inhibitor can be one or more of tazemetostat, 3-deazaneplanocin A (DZNep), EPZ005687, EI1, GSK126, EPZ-6438, GSK343, GSK503, CPI-1205, Constellation Compound 3, +OR-S2, and +UNC1999.

The pharmaceutical compositions of an EZH2 inhibitor or a PARP inhibitor can contain one or more acceptable carriers therefor and optionally other therapeutic ingredients. The carrier(s) is a compound compatible with the other ingredients of the pharmaceutical composition and not deleterious to the patient. The dosage regimen of the pharmaceutical compositions may include any controlled release dosage form of the therapeutically effective amount of an EZH2 inhibitor and the therapeutically effective amount of a PARP inhibitor. In certain embodiments, the controlled release dosage form is an oral dosage form such as, for example, a tablet or capsule. In certain embodiments, one or both of the therapeutically effective amount of an EZH2 inhibitor and the therapeutically effective amount of a PARP inhibitor can be formulated for parenteral administration and can include aqueous or non-aqueous sterile injection solutions. In certain embodiments, the therapeutically effective amount of an EZH2 inhibitor and the therapeutically effective amount of a PARP inhibitor can be formulated for different and separate modes of administration. The EZH2 inhibitor can be administered simultaneously with, prior to, or after administration of a PARP inhibitor or radiotherapy to a patient.

Illustratively, an effective amount of the compositions of this invention ranges from nanogram/kg to milligram/kg amounts for young children and adults. Equivalent dosages for lighter or heavier body weights can readily be determined. The dose should be adjusted to suit the individual to whom the composition is administered and will vary with age, weight and metabolism of the individual. The exact amount of the composition required will vary from subject to subject, depending on the species, age, weight and general condition of the subject, the particular peptide or polypeptide used, its mode of administration and the like. An appropriate amount can be determined by one of ordinary skill in the art using only routine experimentation given the teachings herein. Optimal dosages to be administered may be readily determined by those skilled in the art, and will vary with the particular compound used, the mode of administration, the strength of the preparation, the mode of administration, the number of consecutive administrations within a limited period of time, and the advancement of the disease condition. In addition, factors associated with the particular patient being treated, including patient age, weight, diet and time of administration, will result in the need to adjust dosages.

Embodiments also include evaluation of a group of EZH2 target genes that are susceptible to downregulation upon EZH2 inhibition to determine the course of treatment for a patient. This group of EZH2 target genes include certain genes in the base excision repair (BER) pathway. Embodiments also include methods of identifying patients susceptible to EZH2 inhibitors. One such method includes obtaining a biological sample containing cancer cells from a patient and evaluating the expression of a group of DNA repair genes as a predictor of sensitivity of the cancer cells to EZH2 inhibitors. Using these methods, patients can be identified who will benefit from a combination therapy of EZH2 inhibitors with radiotherapy or a PARP inhibitor, such as olaparib. The EZH2 inhibitors can be used to sensitize cancer cells to DNA damaging agents, such radiotherapy and PARP inhibitors. EZH2 inhibitors may overcome radiotherapy resistance in advanced cancers and improve the efficacy of PARP inhibitors even in BRCA-proficient tumors. The panel of biomarkers disclosed here can be used to evaluate the benefits of these combination therapies in cancer patients. Provided here is a method of assessment of sensitivity of cancer cells to EZH2 inhibitors based on a gene expression signature of certain DNA damage repair proteins. These genes are targeted by EZH2 inhibitors and underlie the tumor-suppressive effects of these EZH2 inhibitors. EZH2 inhibitors can be administered to sensitize cancers, which overexpress EZH2-regulated DNA damage repair genes, to genotoxic agents.

Embodiments include methods of assessing response of cancer cells from a patient to an EZH2 inhibitor. A method includes comparing expression levels of a plurality of biomarkers in a first biological specimen from a patient to expression levels of the plurality of biomarkers in a second biological specimen from the patient, wherein the first biological specimen contains cancer cells and the second biological specimen contains normal control cells of the patient and a change in the expression levels of the plurality of biomarkers indicates a response to one or more EZH2 inhibitors. The plurality of biomarkers includes genes or gene expression products or fragments thereof from one or more of the base excision repair, mismatch repair, Fanconi anemia pathway, homologous recombination, or non-homologous end-joining pathways, as described in the Kyoto Encyclopedia of Genes and Genomes (KEGG). In an embodiment, the plurality of biomarkers includes genes or gene expression products or fragments thereof from the following: CDCA3, CKS2, MUTYH, POLE3, TACC3, RFC4, RFC2, REV3L, POLD4, MLH1, FANCG, FANCF, FANCL, BRCA1, POLE2, BRIP1, PRKDC, POLI, NEIL1, PARP3, POLM, LIG1, BARD1, DCLRE1C, UBE2T, MPG, FEN1, RBBP8, RFC3, BRCC3, FANCD2, RFC5, POLE, POLB, HES1, RAD54L, DNTT, MSH2, POLD1, BRCA2, POLE4, PARP1, MLH3, NEIL3, BLM, RPA2, RPA3, RAD50, NTHL1, LIG4, POLH, XRCC4, NEIL2, RMI2, FANCI, UNG, MSH6, EXO1, ATM, NBN, RMI1, HMGB1, and RAD51C. In an embodiment, the plurality of biomarkers includes genes or gene expression products or fragments thereof from three or more of CDCA3, CKS2, MUTYH, POLE3, TACC3, RFC4, RFC2, REV3L, POLD4, MLH1, FANCG, FANCF, FANCL, BRCA1, POLE2, BRIP1, PRKDC, POLI, NEIL1, PARP3, POLM, LIG1, BARD1, DCLRE1C, UBE2T, MPG, FEN1, RBBP8, RFC3, BRCC3, FANCD2, RFC5, POLE, POLB, HES1, RAD54L, DNTT, MSH2, POLD1, BRCA2, POLE4, PARP1, MLH3, NEIL3, BLM, RPA2, RPA3, RAD50, NTHL1, LIG4, POLH, XRCC4, NEIL2, RMI2, FANCI, UNG, MSH6, EXO1, ATM, NBN, RMI1, HMGB1, and RAD51C. Significant change is measured by determining the mean expression level of the biomarkers from the first biological specimen from a patient and comparing them to the mean expression of the biomarkers in the second biological specimen. A significant increase of mean expression value is defined as higher than mean plus one standard deviation of expression distribution between the specimens. The exact threshold and units would be dependent on the detection platform used.

Embodiments include a set of reagents to measure the levels of the plurality of biomarkers in a specimen, wherein the biomarkers are one or more of CDCA3, CKS2, MUTYH, POLE3, TACC3, RFC4, RFC2, REV3L, POLD4, MLH1, FANCG, FANCF, FANCL, BRCA1, POLE2, BRIP1, PRKDC, POLI, NEIL1, PARP3, POLM, LIG1, BARD1, DCLRE1C, UBE2T, MPG, FEN1, RBBP8, RFC3, BRCC3, FANCD2, RFC5, POLE, POLB, HES1, RAD54L, DNTT, MSH2, POLD1, BRCA2, POLE4, PARP1, MLH3, NEIL3, BLM, RPA2, RPA3, RAD50, NTHL1, LIG4, POLH, XRCC4, NEIL2, RMI2, FANCI, UNG, MSH6, EX01, ATM, NBN, RMI1, HMGB1, RAD51C, or measurable fragments thereof. In an embodiment, the set of reagents to measure the levels of the plurality of biomarkers in a specimen, wherein the biomarkers are one or more of CDCA3, CKS2, MUTYH, POLE3, TACC3, or measurable fragments thereof. The biomarkers can be used in various combinations for diagnosis. The disclosure provides biomarkers, methods, devices, reagents, systems, and kits for assessing response of cancer cells from a patient to an EZH2 inhibitor or the therapeutic efficacy of an EZH2 inhibitor for treatment of a patient afflicted with cancer.

Embodiments include methods of assessing response of cancer cells from a patient to an EZH2 inhibitor or therapeutic efficacy of an EZH2 inhibitor. One such method includes comparing expression levels of a plurality of biomarkers (as provided in paragraph [0038]) in a first biological specimen from a patient to expression levels of the plurality of biomarkers in a second biological specimen from the patient, wherein the second biological specimen is obtained by exposing the first biological specimen to an EZH2 inhibitor and a change in the expression levels of the plurality of biomarkers indicates sensitivity of cancer cells from a patient to the EZH2 inhibitor or therapeutic efficacy of the EZH2 inhibitor. In an embodiment, the first biological specimen includes prostate cancer cells.

Another method includes assessing response of cancer cells from a patient to multiple EZH2 inhibitors by evaluating expression levels of a plurality of the biomarkers, as provided in paragraph [0038]. Another method includes obtaining a biological sample containing cancer cells from a patient and evaluating the expression of a group of DNA damage repair genes (as provided in paragraph [0038]) to measure sensitivity of the cancer cells to EZH2 inhibitors, either as monotherapy or in combination with DNA damaging agents.

A method of assessing response of cancer cells from a patient to an EZH2 inhibitor includes comparing expression levels of a plurality of biomarkers in a first biological specimen from a patient to expression levels of the plurality of biomarkers in a second biological specimen from a control subject who does not have cancer, wherein a change in the expression levels of the plurality of biomarkers between the two specimens indicates a response to one or more EZH2 inhibitors. The second biological specimen can contain normal cells from the same tissue in a control subject who does not have cancer. Significant change is measured by determining the mean expression level of the biomarkers from the cancer cells from a patient and comparing them to the mean expression of the biomarkers in normal control tissues. A significant increase of mean expression value is defined as higher than mean plus one standard deviation of expression distribution from normal tissues. The exact threshold and units would be dependent on the detection platform used.

It will be noted that embodiments of the method of treating cancer by administering an EZH2 inhibitor and a PARP-1 inhibitor are also taken as embodiments of an EZH2 inhibitor and a PARP-1 inhibitor or their pharmaceutically acceptable derivatives for use in the treatment of cancer or use of an EZH2 inhibitor and a PARP-1 inhibitor or their pharmaceutically acceptable derivatives in the manufacture of a medicament for treating cancer and reciprocals thereof. It will also be noted that embodiments of the method of treating cancer, an EZH2 inhibitor and a PARP-1 inhibitor or their pharmaceutically acceptable derivatives for use in the treatment of cancer, or an EZH2 inhibitor and a PARP-1 inhibitor or their pharmaceutically acceptable derivatives in the manufacture of a medicament for treating cancer are also taken as embodiments of the pharmaceutical composition, pharmaceutical formulation, or pharmaceutical kit.

The following non-limiting examples are provided in order to further illustrate the various embodiments.

### EXAMPLES

### Example 1—EZH2 Inhibitors Suppress the Proliferation of Androgen Receptor (AR)-Positive Prostate Cancer Cells

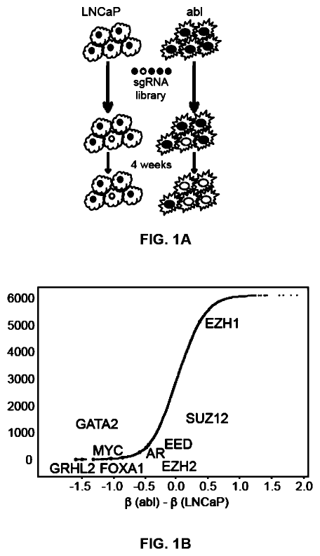

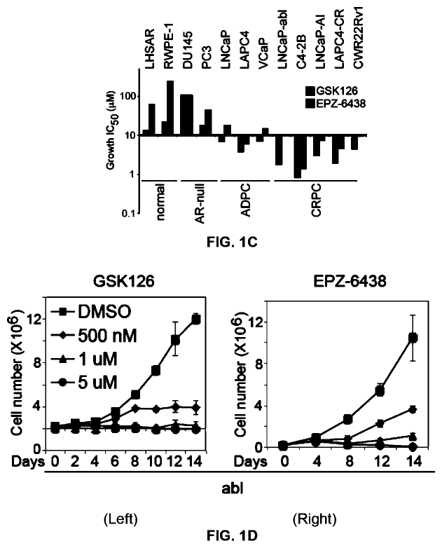

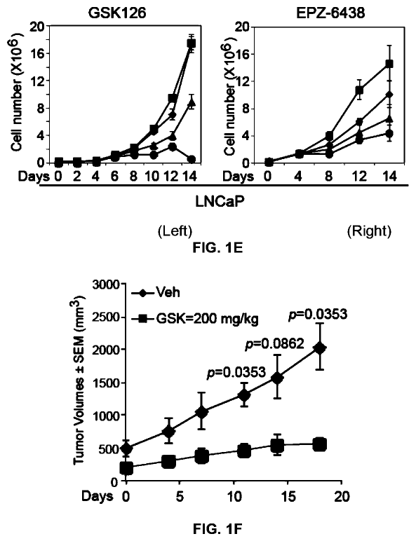

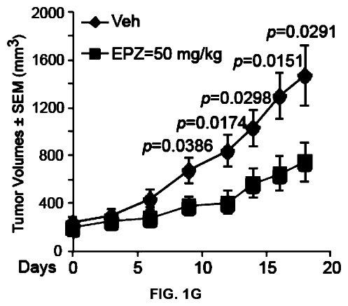

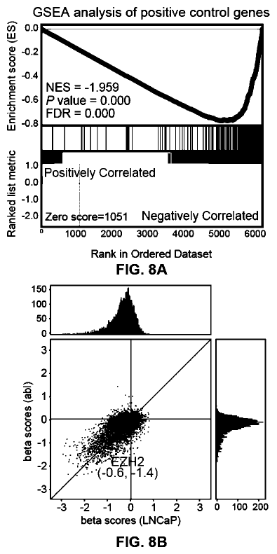

To determine the gene(s) essential for the sustained proliferation of androgen-dependent and castration resistant prostate cancer (CRPC) cells in an unbiased manner, CRISPR-Cas9 knockout screening was conducted in both parental, hormone-dependent LNCaP cells and its hormone-refractory counterpart LNCaP-abl (abl) cells under their respective proliferating conditions (FIG. 1A). FIG. 1A is a diagrammatic representation of the work flow of CRISPR knockout screens in LNCaP and abl cells. Positive control genes that are known to be required for cell proliferation in general were strongly selected, suggesting that the screens could reliably identify new essential genes (FIG. 8A). FIG. 8A is a graph of gene set enrichment analysis (GSEA) result showing positive sgRNAs that are known to regulate cell proliferation were indeed negatively selected in both LNCaP and abl cells. MAGeCK was used to analyze the CRISPR screens, which assigns a beta score to each gene to approximate change of CRISPR guide DNA abundance. Therefore, in the cells grown for 4 weeks compared with those on day 0, more negative beta score represents higher dependency of the target gene on cell growth. Most genes possessed similar beta scores between these two prostate cancer cell lines (FIG. 8B). FIG. 8B is a graph of distributions of beta scores from the CRISPR-Cas9 knockout screening in LNCaP (x-axis) and abl (y-axis) cells. Position of EZH2 was indicated by red dots. Numbers in the parentheses, beta scores in LNCaP (the first values) and abl (the second values).

Genes that are more essential for the androgen-independent growth of CRPC cells were evaluated by comparing the beta scores between LNCaP and abl. Several transcription factors that are well known for their prominent roles in CRPC, such as AR, FOXA1, and MYC, were consistently identified. Interestingly, EZH2 was one of the top hits that possessed much stronger dependency in abl cells than in LNCaP (FIG. 1B), while EZH1, another mammalian homolog of Drosophila Enhancer of Zeste, was not required for either cell lines. FIG. 1B is a graphical representation of the difference of gene essentialities, represented by beta (β) scores, between abl and LNCaP [β(abl)-β(LNCaP)] for each individual gene. Positions of representative genes were indicated by red dots.

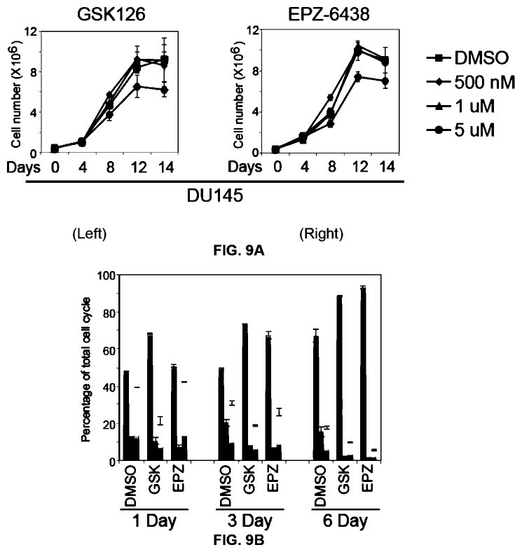

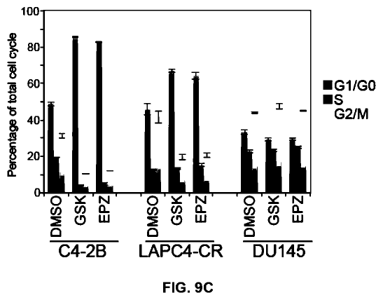

Genetic inhibition of EZH2 suppressed growth of CRPC cells better than that of androgen-dependent prostate cancer cells. As EZH2 is a druggable enzyme with available small-molecule inhibitors, the efficacy of EZH2-targeting drugs was assessed in prostate cancer cells. Two compounds, GSK126 and EPZ-6438, were tested in a panel of human prostate cell lines, including two benign prostate epithelial cells, two AR-null prostate cancer cells, and eight AR-positive prostate cancer cells. Only malignant cells with intact AR signaling, especially the hormone-refractory lines, were sensitive to both inhibitors. FIG. 1C is a graphical representation of the IC50 for GSK126 (red bars) and EPZ-6438 (blue bars) in a panel of prostate normal and cancer cell lines after 6 days of treatment. Cells were grouped based on the basic characteristics. ADPC is androgen-dependent prostate cancer, and CRPC is castration-resistant prostate cancer. Concentrations as low as 500 nM of the EZH2 inhibitors (EZH2i) greatly retarded the growth of castration-resistant abl cells. FIG. 1D is a graphical representation of the effects of EZH2 inhibitors (left panel for GSK126, and right panel for EPZ-6438) on cell growth over time in abl cells with indicated concentrations of the compounds. Relatively higher doses of EZH2i were required to suppress the androgen-dependent LNCaP cells. FIG. 1E is a graphical representation of the effects of EZH2 inhibitors (left panel for GSK126, and right panel for EPZ-6438) on cell growth over time in LNCaP cells with indicated concentrations of the compounds. The inhibitory effect of EZH2i was minimal in AR-null DU145 (FIG. 9A; GSK126 treatment in the left panel and EPZ-6438 in the right panel). This was consistent with a previous report that neither PC3 nor DU145 requires EZH2 for continued growth. Cell cycle analysis showed that EZH2i induced G0-G1 arrest in responsive CRPC cell lines within 3 days of the drug treatment (FIG. 9B), but no cytostatic effect was observed in the unresponsive DU145 cells (FIG. 9C). FIG. 9B is the box plot showing cell cycle analysis of abl cells with the treatment of vehicle (DMSO), 5 μM GSK126 (GSK) or 5 μM EPZ-6438 (EPZ) over indicated days. FIG. 9C is the box plot showing prostate cancer cells (C4-2B, LAPC4-CR and DU145) after being treated with vehicle (DMSO), 5 μM GSK126 (GSK) or 5 μM EPZ-6438 (EPZ) for 3 days, and then subjected to propidium iodide staining followed by flow cytometry analysis.

Inhibitors of EZH2 methyltransferase activity showed potent inhibitory effects in prostate cancer cells, especially the castration resistant ones. The EZH2i effect on CRPC cell growth was evaluated in vivo, subcutaneous xenografts of the hormone refractory CWR22Rv1 cells in castrated mice were treated with either GSK126 (FIG. 1F) or EPZ-6438 (FIG. 1G). FIG. 1F is a growth curve of xenograft tumors in castrated nude mice injected with CWR22Rv1 cells receiving vehicle (Veh) or GSK126, at the indicated doses. FIG. 1G is a growth curve of xenograft tumors in castrated nude mice injected with CWR22Rv1 cells receiving vehicle (Veh) or EPZ-6438, at the indicated doses. Both compounds significantly retarded tumor growth following 21 days of treatment. Thus, EZH2 was identified as a therapeutic target for prostate cancer and that EZH2 inhibitors would benefit patients with AR-positive, metastatic, hormone-refractory tumors.

### Example 2—EZH2 Inhibition Induces Specific Gene Signatures in Sensitive CRPC Cells

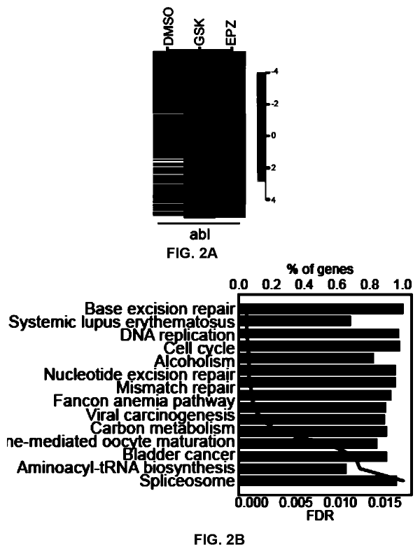

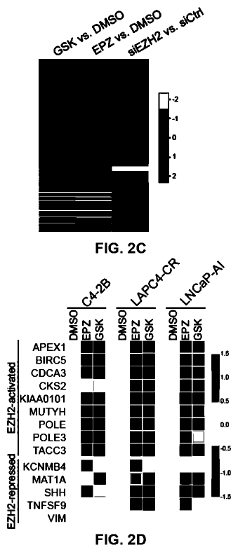

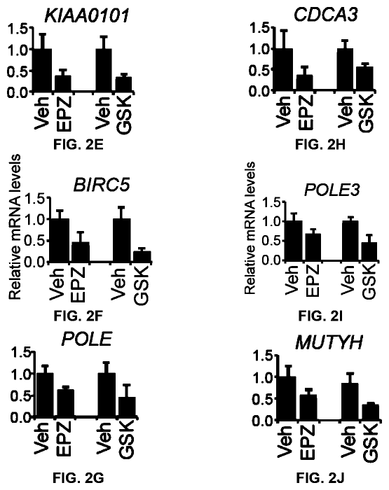

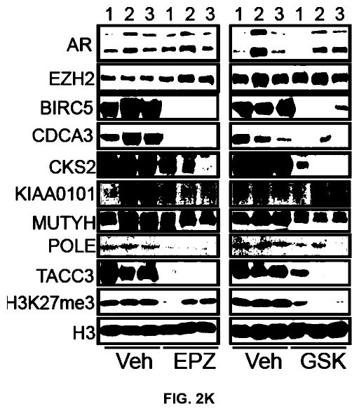

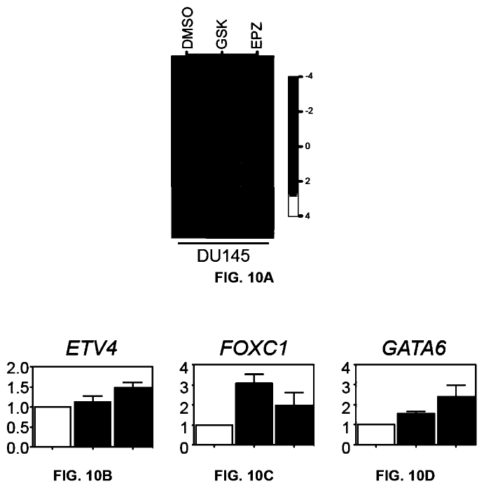

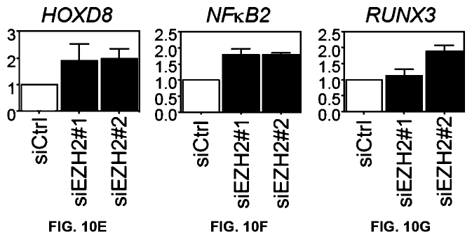

To investigate the mechanisms underlying the action of EZH2 inhibitors in sensitive prostate cancer cells, the gene expression pattern of abl cells was profiled upon the treatment with GSK126 or EPZ-6438. FIG. 2A is a heat map of differential gene expression patterns in abl cells being treated with vehicle (DMSO), 5 μM GSK126 (GSK) or 5 μM EPZ-6438 (EPZ) for 72 hrs. Both compounds induced very similar transcriptional changes, indicating likely on-target genetic effects. Interestingly, a large number of genes were significantly downregulated instead of being de-repressed, which is in contrast to susceptible DLBCL cells where EZH2 inhibitors were reported to induce a robust transcriptional activation. Genes that were commonly downregulated by both EZH2i in abl cells were significantly enriched in DNA damage response (DDR), such as various DNA damage repair pathways, cell cycle regulation and DNA replication process. FIG. 2B is an illustration of overrepresented functional annotations of genes that were significantly downregulated upon the treatment with EZH2 inhibitors in abl cells from Gene Set Enrichment Analysis. Blue bars represent the percentage of genes in each specific functional category. The red line represents values of the false discovery rate (FDR) for the particular GO term. There were no significant functional annotations for the upregulated genes in abl cells. Two analyses were carried out to rule out the possible off-targeted effect of EZH2i on gene regulation. First, transcriptional profiles were compared in abl being treated with either EZH2i or in which EZH2 had been silenced using RNA interference (RNAi). Pharmacologic and genetic inhibition of EZH2 shared highly similar gene expression patterns. FIG. 2C is a heat map of differential genes induced by EZH2 inhibitors (GSK, GSK126; EPZ, EPZ-6438) comparing to vehicle control (GSK vs. DMSO and EPZ vs. DMSO) or EZH2 knockdown comparing to control siRNA (siEZH2 vs. siCtrl) in abl cells. Second, in EZH2i-insensitive DU145 cells, GSK126 and EPZ-6438 largely de-repressed genes (FIG. 10A) which, consistent with the de-repressed genes upon EZH2 silencing, recapitulates the suppressive function of EZH2 as an H3K27 methyltransferase. FIG. 10A is the heat map of differential genes in DU145 cells, which were treated with vehicle (DMSO), 5 μM GSK126 (GSK) or 5 μM EPZ-6438 (EPZ) for 72 hrs. FIGS. 10B-10G are bar plots demonstrating the effect of EZH2 knockdown in DU145 cells on the expression of ETV4 (FIG. 10B), FOXC1 (FIG. 10C), GATA6 (FIG. 10D), HOXD8 (FIG. 10E), NFB2 (FIG. 10F), and RUNX3 (FIG. 10G).

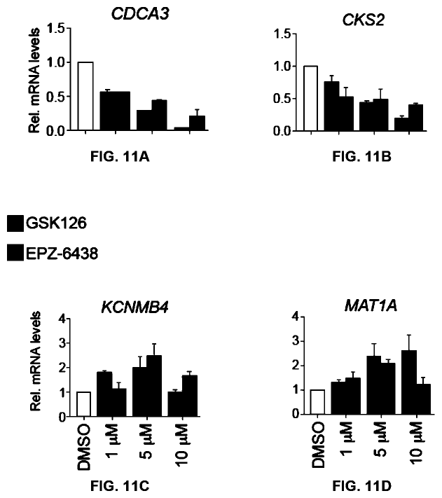

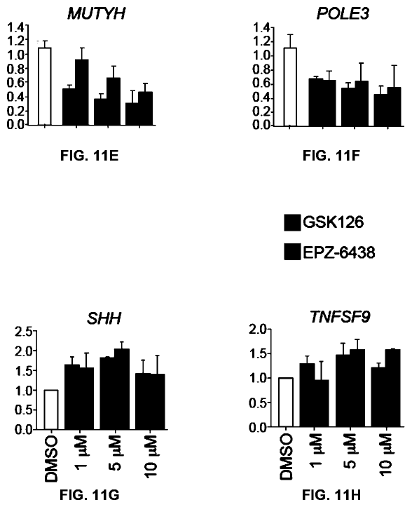

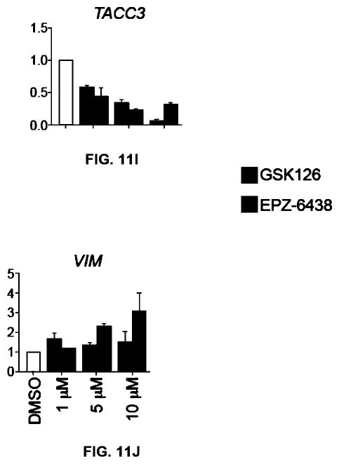

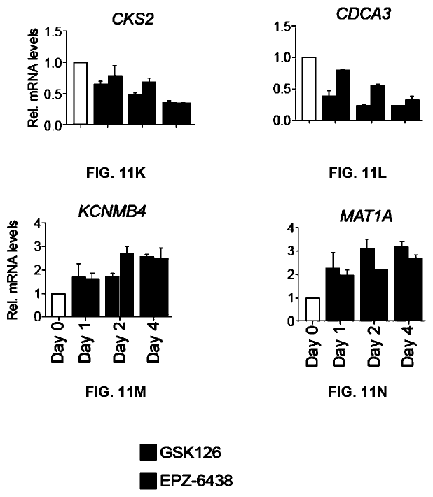

Several differentially expressed genes were validated to show that EZH2i suppressed their expression in a dose-dependent and time-dependent manner. FIGS. 11A-11J are bar plots demonstrating the effect of EZH2 inhibitors (GSK126 or EPZ-6438) on expression of the following genes in abl cells at different concentrations for 72 hrs: CDCA3 (FIG. 11A), CKS2 (FIG. 11B), KCNMB4 (FIG. 11C), MAT1A (FIG. 11D), MUTYH (FIG. 11E), POLE3 (FIG. 11F), SHH (FIG. 11G), TNFSF9 (FIG. 11H), TACC3 (FIG. 11I), and VIM (FIG. 11J). FIGS. 11K-11T are bar plots demonstrating the effect of EZH2 inhibitors (GSK126 or EPZ-6438) on expression of the following genes in abl cells at 5 μM for indicated days: CKS2 (FIG. 11K), CDCA3 (FIG. 11L), KCNMB4 (FIG. 11M), MAT1A (FIG. 11N), MUTYH (FIG. 11O), POLE3 (FIG. 11P), TACC3 (FIG. 11Q), SHH (FIG. 11R), TNFSF9 (FIG. 11S), and VIM (FIG. 11T).

The expression of these genes was examined in three other EZH2i-sensitive CRPC cell lines: C4-2B, LAPC4-CR and LNCaP-AI. FIG. 2D is a heat map of quantitative real-time RT-qPCR results showing changes in mRNA levels of selected gene in three prostate cancer cell lines (C4-2B, LAPC4-CR, and LNCaP-AI). Cells were treated with vehicle (DMSO), 5 μM GSK126 (GSK) or 5 μM EPZ-6438 (EPZ) for 72 hrs. EZH2-activated genes and EZH2-repressed genes are grouped together. While GSK126 and EPZ-6438 upregulated EZH2-repressed genes to some extent, EZH2i decreased expression of EZH2-activated genes more consistently in these cells. These findings were validated in CWR22Rv1 xenografts, demonstrating consistent decrease in mRNA (FIGS. 2E-2J) and protein (FIG. 2K) levels of EZH2-activated genes by either compound. Taken together, these results indicated that EZH2 inhibitors target the methyltransferase activity of EZH2, and yet they downregulate a group of EZH2-activated genes in CRPC cells. FIGS. 2E-2J are graphical representation of the mRNA expression of the following genes in xenograft tumor tissues from control mice (Veh) or mice treated with GSK126 (GSK) or EPZ-6438 (EPZ): KIAA0101 (FIG. 2E), BIRC5 (FIG. 2F), POLE (FIG. 2G), CDCA3 (FIG. 211), POLE3 (FIG. 21), and MUTYH (FIG. 2J). FIG. 2K is a graphical representation of the protein levels of selected genes in xenograft tumor tissues from control mice (Veh) or mice treated with GSK126 (GSK) or EPZ-6438 (EPZ). Numbers, triplicates of samples from control or treatment group.

### Example 3—EZH2i-Resistance Mutations Rescue the Effects of EZH2 Inhibitors on Gene Expression and Cell Growth

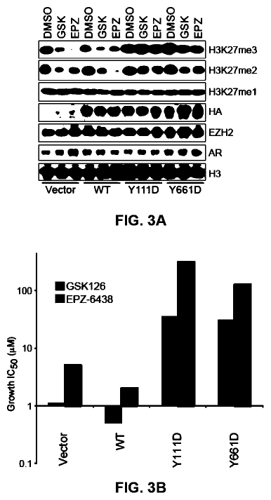

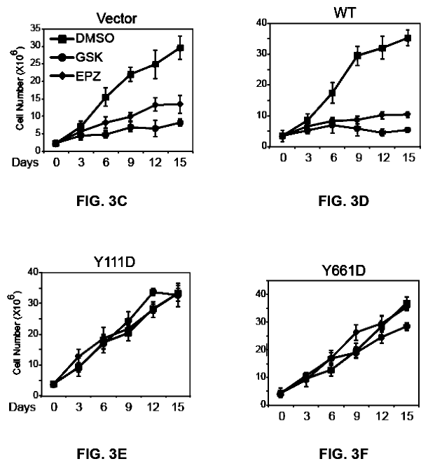

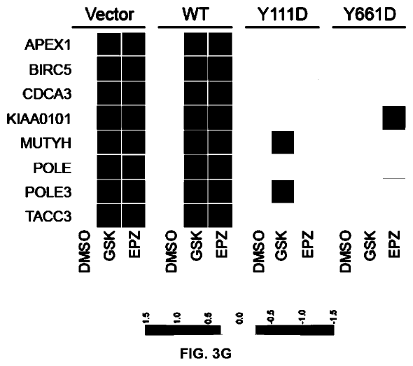

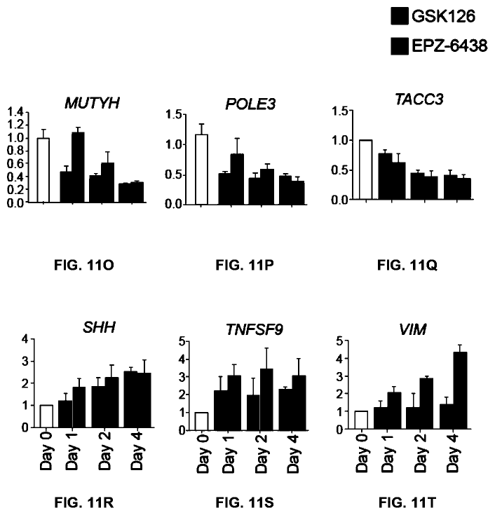

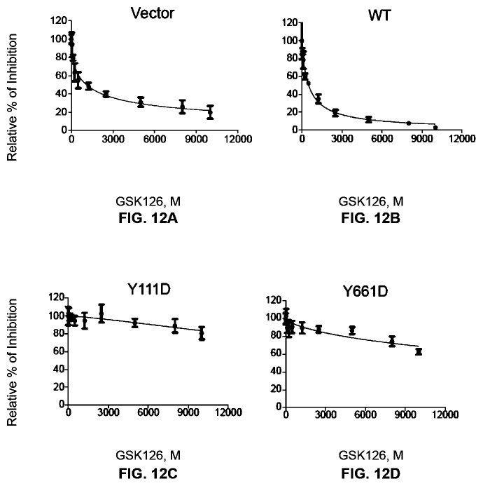

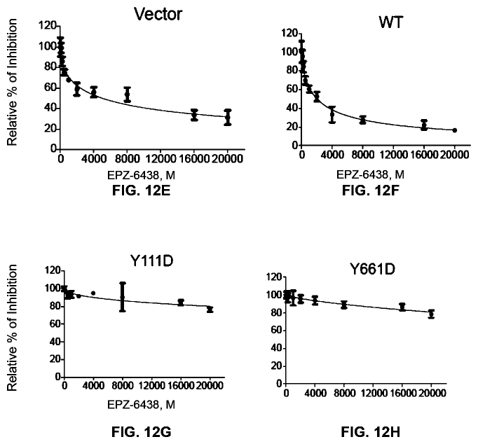

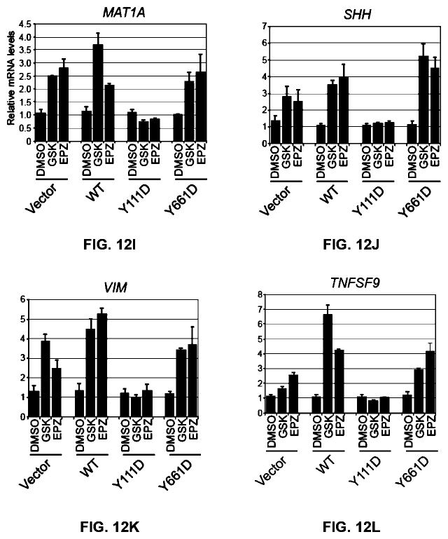

Several resistance mutations in EZH2, including Y111D and Y661D, were identified in DLBCL clones that became refractory to EZH2i. These resistance mutants regain methyltransferase activity in the presence of EZH2i, and thus offer a genetic means to evaluate the targeted action of EZH2i. Endogenous EZH2 in abl were replaced with these mutants, and the responses of both parental and mutant cell lines to EZH2i were evaluated by assessing H3K27 methylation, cell proliferation, and gene expression. When either Y111D or Y661D was expressed, EZH2i-induced reduction of H3K27me3 level was dramatically alleviated. FIG. 3A is a photographic image of Western blots of H3K27 methylation levels in abl cells that were replaced with the control (Vector), the wild type (WT) or EZH2 mutants bearing different point mutations (Y111D or Y661D). Cells were treated with DMSO or EZH2 inhibitors (GSK, GSK126; EPZ, EPZ-6438) for 72 hrs. This was especially notable in the case of Y111D, which completely abolished the effect of EZH2i on H3K27me3. In cell growth assays, EZH2i failed to suppress the growth of abl cells expressing Y111D or Y661D (FIG. 3B, FIGS. 12A-12H), even after incubation with the drugs for up to 15 days (FIGS. 3C-3F). FIG. 3B is a graphical representation of the IC50 values for two EZH2 inhibitors in abl cells expressing the control (Vector), the wild-type EZH2 (WT) or EZH2 mutants bearing different point mutations (Y111D or Y661D). Cells were incubated with EZH2 inhibitors (GSK126, red bars; EPZ-6438, blue bars) for 6 days and then collected for direct counting after trypan blue staining. FIG. 3C is a graphical representation of effects of EZH2 inhibitors (GSK referring to GSK126, and EPZ referring to EPZ-6438) on the growth of abl cells expressing the control (Vector) construct at indicated time points. These results demonstrated that mutations of EZH2 that render it resistant to EZH2i in DLBCL also confer resistance in prostate cancer cells. FIG. 3D is a graphical representation of effects of GSK126, and EPZ-6438) on the growth of abl cells expressing the wild-type EZH2 (WT) at indicated time points. FIG. 3E is a graphical representation of effects of GSK126 and EPZ-6438 on the growth of abl cells expressing the EZH2 mutant—Y111D—at indicated time points. FIG. 3F is a graphical representation of effects of GSK126 and EPZ-6438 on the growth of abl cells expressing the EZH2 mutant—Y661D—at indicated time points. FIGS. 12A-12D are graphical representations of abl cells expressing the control (Vector) (FIG. 12A), the wild-type EZH2 (WT) (FIG. 12B), EZH2 mutant (Y111D) (FIG. 12C), and EZH2 mutant (Y661D) (FIG. 12D) when incubated with indicated concentrations of GSK126 for 6 days. Cell numbers were counted at the end point after trypan blue staining, and normalized to that under vehicle condition, which was considered as 100%. FIGS. 12E-12H are graphical representations of abl cells expressing the control (Vector) (FIG. 12E), the wild-type EZH2 (WT) (FIG. 12F), EZH2 mutant (Y111D) (FIG. 12G), and EZH2 mutant (Y661D) (FIG. 1211) when incubated with indicated concentrations of EPZ-6438 (B) for 6 days. Cell numbers were counted at the end point after trypan blue staining, and normalized to that under vehicle condition, which was considered as 100%.

Intriguingly, both Y111D and Y661D rescued the expression of EZH2-activated genes in the presence of EZH2i (FIG. 3G). FIG. 3G is a diagrammatic representation of the expression of EZH2-activated genes as detected by RT-qPCR in abl cells that were replaced with the control (Vector), the wild-type EZH2 (WT) or the mutants in the presence of vehicle (DMSO) or 5 μM EZH2 inhibitors (GSK, GSK126; EPZ, EPZ-6438) for 3 days. Interestingly, this rescue was less evident for EZH2-repressed genes (FIGS. 12I-12L). These findings provide strong support for the conclusion that EZH2 inhibitors abrogated prostate cancer cell growth through specific blockade of EZH2 functions. They also support that genes transactivated by EZH2 might predominantly mediate the action of EZH2 inhibitors in CRPC. FIGS. 12I-12L are graphical representations of expression of the following EZH2-repressed genes upon EZH2 inhibitor treatment (GSK126 or EPZ-6438) in abl cells expressing the control (Vector), the wild-type EZH2 (WT), EZH2 mutant (Y111D), and EZH2 mutant (Y661D): MATA1A (FIG. 12I), SHE (FIG. 12J), VIM (FIG. 12K), and TNFSF9 (FIG. 12L).

### Example 4—EZH2 Inhibitors Decrease Global H3K27Me3 Signal on Chromatin Regardless of Cellular Response to the Compounds

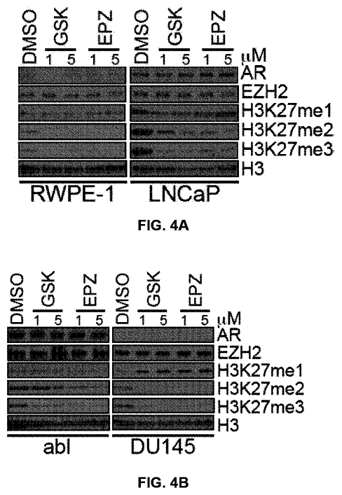

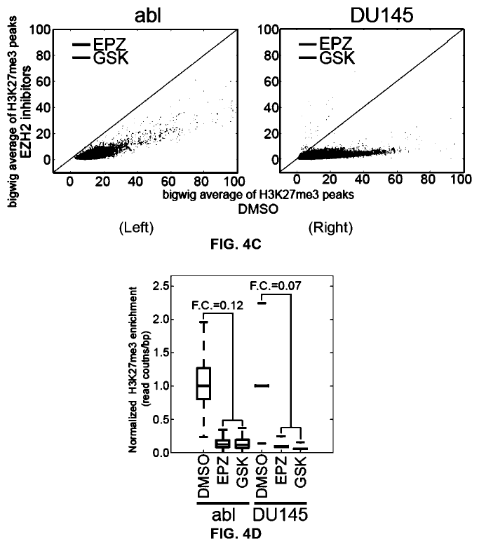

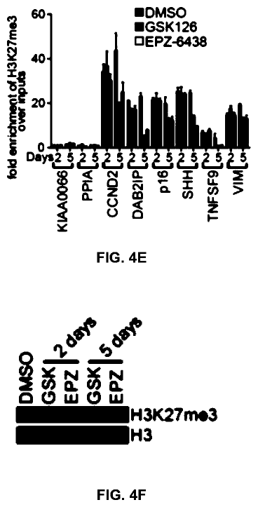

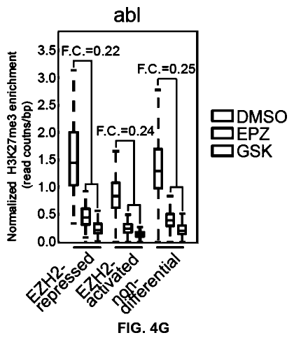

In view of the canonical function of EZH2 in catalyzing H3K27me3, the mechanisms of how repressive chromatin mark contribute to the growth and gene inhibitory effects of EZH2i were investigated. All of the tested prostate cell lines demonstrated reduced total H3K27 di- and tri-methylation levels in a dose-dependent manner with either compound (FIGS. 4A-4B), which is in contrast with their distinct cellular growth responses to EZH2i. Discrepancies between EZH2i-induced H3K27me3 reduction and cell insensitivity to EZH2i were also reported in lymphoma cells. FIGS. 4A and 4B are photographic images of the H3K27 methylation levels in prostate cell lines with the treatment of vehicle (DMSO), GSK126 (GSK) or EPZ-6438 (EPZ) at specified doses (1 or 5 μM final concentration).

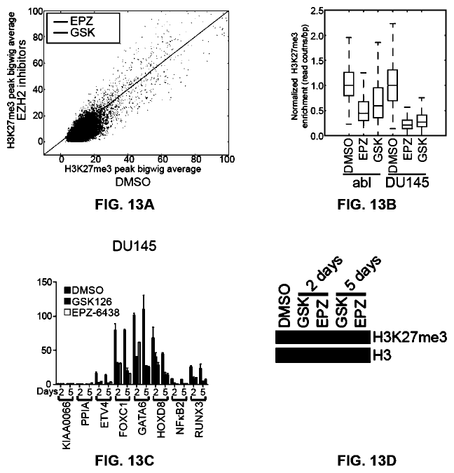

To accurately evaluate the locus-specific changes of H3K27me3 on chromatin, the ChIP-Rx method was adopted, which uses a “SPIKE-IN” strategy to quantify genome-wide histone modification relative to a reference epigenome with defined quantities. Canonical normalization methods such as using the total sequencing reads showed moderate H3K27me3 changes after cells were treated with EZH2i (FIG. 13A), while normalization to the reference epigenome showed pronounced H3K27me3 reductions (FIG. 4C). FIG. 13A is a representation of the intensity of each H3K27me3 peak as compared between control condition (DMSO, x-axis) and treatment condition (EZH2 inhibitors, y-axis) in abl cells, using reads per million methods. FIG. 4C is a set of scatter plots of H3K27me3 peak signals, after being normalized to the Drosophila reference epigenome, in abl cells (left panel) and DU145 cells (right panel) under control condition (x-axis) or after the treatment with EZH2 inhibitors (y-axis). In the EZH2i-insensitive DU145 cells, we noticed a similar or even more robust EZH2i-induced decrease of H3K27me3 intensity than in abl (FIG. 4D and FIG. 13B). FIG. 4D is a graphical representation of the EZH2 inhibitor-induced changes in H3K27me3 levels between abl and DU145 cells after SPIKE-IN normalization (GSK referring to GSK126, and EPZ referring to EPZ-6438; F.C., fold change). FIG. 13B is a representation of the H3K27me3 peak enrichment under the conditions of vehicle (DMSO), 5 μM GSK126 (GSK) or 5 μM EPZ-6438 (EPZ) as normalized using canonical method and then compared between abl and DU145 cells.

This implies that reduction of H3K27me3 alone does not confer growth sensitivity to EZH2i. Indeed, a moderate decrease was detected in overall H3K27me3 amount and its signal on chromatin in abl cells as early as 2 days of EZH2i treatment (FIGS. 4E and 4F), and DU145 cells showed a marked loss of the histone modification within the same time frame (FIGS. 13C-13D). FIG. 4E is a graphical representation of the direct ChIP-qPCR results of H3K27me3 at selected chromatin regions after abl cells were treated with control (DMSO) or EZH2 inhibitors (GSK126 or EPZ-6438) for indicated number of days. KIAA0066 and PPIA are negative controls. FIG. 4F is a photographic image of an immunoblot of H3K27me3 protein levels in the corresponding ChIP samples (GSK referring to GSK126, and EPZ referring to EPZ-6438). FIG. 13C is a graphical representation of direct ChIP-qPCR of H3K27me3 as performed and detected at selected chromatin regions in DU145 cells. FIG. 13D is a photographic image of the immunoblot of H3K27me3 protein levels in the corresponding ChIP samples.

To find any functional significance of EZH2i-triggered H3K27me3 alterations in abl cells, changes of the repressive histone mark were associated with differential gene expression upon compound treatment (FIG. 4G). FIG. 4G is a graphical representation of the SPIKE-IN normalized signals of H3K27me3 peaks around genes that were upregulated (EZH2-repressed), downregulated (EZH2-activated) or showed no differences (non-differential) upon the treatment with EZH2 inhibitors. Intensities of the histone mark under either control (DMSO) or treatment condition (GSK referring to GSK126, and EPZ referring to EPZ-6438) were plotted. Although basal level of H3K27me3 was noticeably higher at the promoter regions of EZH2i-upregulated genes, there were no differences regarding the extent of H3K27me3 decrease among EZH2-repressed, EZH2-activated or EZH2-indifferent genes. This result suggests that while the steady status of H3K27me3 is associate the silenced transcription of downstream targets, the fluctuation of H3K27me3 signals does not always lead to immediate transcriptional changes of nearby genes. Taken together, these results suggest that H3K27 methylation, a readout of the polycomb repressive function of EZH2, may not be the determining factor of transcriptional changes or cellular responses to EZH2i.

### Example 5-A Core Gene Signature in Regulation of DNA Damage Repair is Essential for the Growth-Inhibitory Effects of EZH2 Inhibitors in Cancer

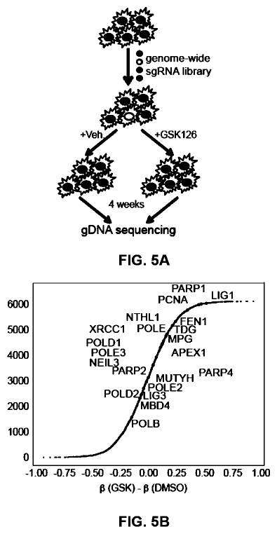

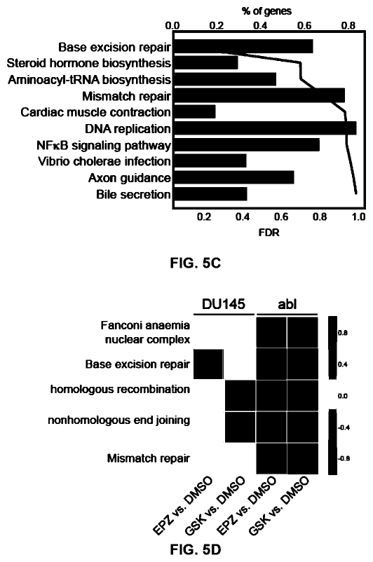

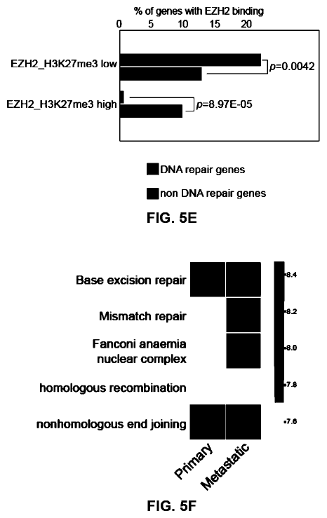

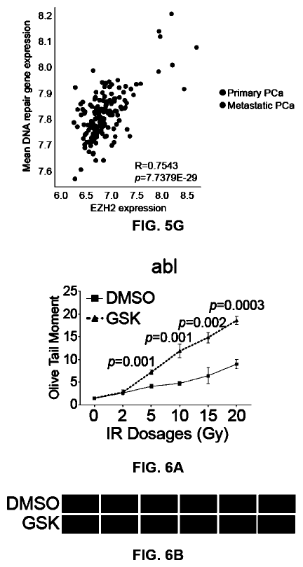

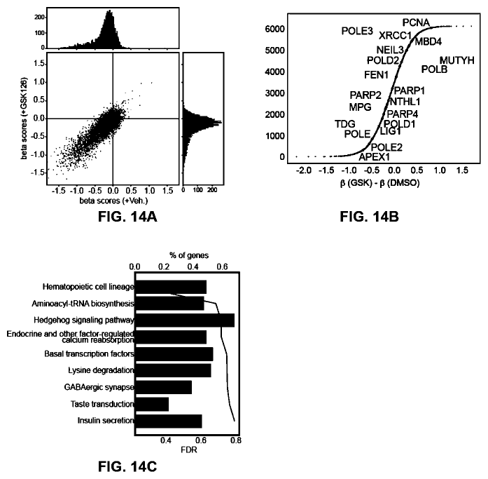

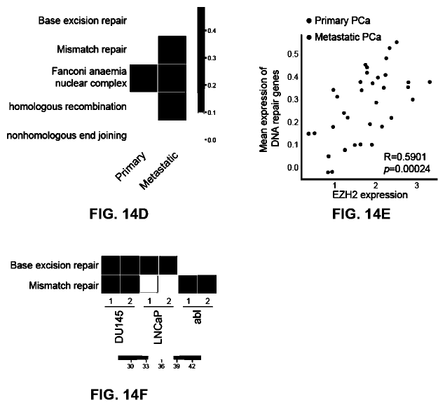

CRISPR-Cas9 knockout screens were conducted in abl with or without GSK126 treatment. FIG. 5A is a diagrammatic representation of the work flow of CRISPR-Cas9 knockout screening, which targets 6,000 cancer-related genes in LNCaP and abl cells in the presence of vehicle or GSK126 (EZH2 inhibitor) for four weeks. As a control, the CRISPR screens were performed in LNCaP in parallel. Most genes showed similar beta scores, a measure of dependency using MAGeCK, between treatment and control conditions in the two cell lines (FIG. 14A). FIG. 14A is a graphical representation of distribution of the beta scores from the CRISPR-Cas9 knockout screening in LNCaP cells under control condition (+Veh., x-axis) and treatment condition (+GSK126, y-axis). To search for genes that mediate specifically the biological effects of GSK126 in CRPC, Ap is defined as the difference in beta scores between treatment (+GSK126) and control (+vehicle) groups. Knockout of genes crucial for the growth-inhibitory effect of EZH2i may render clones resistant to the compounds. Therefore, genes with positive Δβ (i.e., higher beta scores in treatment condition than control condition) are required for EZH2i activity. This analysis identified a group of genes specifically in abl cells, majority of which function in DNA damage repair. FIG. 5B is a graphical representation of the distribution of delta beta (Δβ) scores, defined as beta scores under treatment (+GSK126) condition minus beta scores under control (+Veh.) condition [β(GSK)-β(DMSO)]. Representative DNA repair genes were indicated by red dots.

However, these genes were not selected from the CRISPR-Cas9 screening in LNCaP (FIG. 14B). FIG. 14B is a graphical representation of the difference of gene essentialities, represented by beta (β) scores, between vehicle treatment (DMSO) and EZH2 inhibitor treatment (GSK) for each individual gene. Positions of representative genes were indicated by red dots. Gene set enrichment analysis (GSEA) of genes with positive Δβ values in abl confirmed the significant enrichment of DNA repair processes, especially the base excision repair (BER) pathway (FIG. 5C), while similar analyses of genes with positive Δβ values in LNCap did not find functional enrichment (FIG. 14C). FIG. 5C is a graphical representation of the gene set enrichment analysis of genes with positive delta beta scores (Δβ) in CRISPR-Cas9 knockout screening in abl cells. Blue bars, percentage of genes in each specific functional category; red line, values of the false discovery rate (FDR) for the particular gene ontology term. FIG. 14C is a graph showing functional annotations that were enriched in genes with positive delta beta scores from CRISPR-Cas9 knockout screening in LNCaP cells. Thus far, CRISPR screens suggested that DNA repair genes are directly targeted by EZH2i, and therefore indispensable for the biological effects of EZH2-targeting drugs in prostate cancer cells.

Indeed, EZH2i treatment significantly downregulated expressions of multiple DNA damage repair pathways in abl, but not in DU145 (FIG. 5D). FIG. 5D is a heat map showing differential expression (log-transformed) of genes involved in the specified DNA damage repair pathways upon treatment with EZH2 inhibitors. Transcript levels of these genes in abl and DU145 cells were compared between treatment groups and control group (EPZ vs. DMSO and GSK vs. DMSO). They represent direct targets of EZH2, as EZH2 binding is significantly enriched within ±1 kb of transcription start sites (TSSs) of these genes (FIG. 5E). FIG. 5E is a graphical representation of the percentages of genes containing two types of EZH2 chromatin binding (EZH2_H3K27me3 low, EZH2 binding sites with low or no H3K27me3 signals; EZH2_H3K27me3 high, EZH2 binding sites with high H3K27me3 signals) within 1 kb around their transcriptional start sites. Blue bars, genes involved in DNA damage repair pathways; rose red bars, genes not involved in DNA damage repair. Interestingly, this binding enrichment was only observed for EZH2 binding peaks with low or even no H3K27me3 enrichment, whereas H3K27me3-associated EZH2 peaks were excluded from the promoter regions near these genes. This is in line with the conclusion that H3K27me3 is irrelevant to the activity of EZH2i in CRPC. To further validate the clinical significance of DNA damage repair pathways, their transcript levels in two independent prostate cancer cohorts were retrieved, and they were significantly elevated in metastatic CRPC compared to primary prostate tumors (FIG. 5F and FIG. 14D). FIG. 5F is a graphical representation of the heat map showing expression of DNA repair genes in primary or metastatic prostate cancer. Genes were categorized into different DNA damage repair pathways according to their functions. FIG. 14D is a heat map showing expression of DNA damage repair genes in an independent prostate cancer cohort containing primary or metastatic tumors. Genes were categorized into different DNA repair pathways according to their functions. In addition, expression of these genes shows very strong positive correlation with that of EZH2, both displaying much higher levels in metastatic advanced prostate cancer (FIG. 5G and FIG. 14E). FIG. 5G is a graphical representation of the expression correlation between EZH2 and DNA repair genes in a prostate cancer cohort. Each dot represents a clinical sample, with primary prostate cancer (PCa) samples colored in blue while the metastatic ones colored in yellow. FIG. 14E is a dot plot showing expression correlation between EZH2 and DNA damage repair genes in an independent prostate cancer cohort. Each dot represents a clinical case, and all the primary prostate cancer (PCa) were colored in blue, while the metastatic ones were colored in yellow.

Expression of these genes in prostate cancer cells, especially genes in BER and mismatch repair pathways, is correlated with the cellular sensitivity to EZH2i, which is the lowest in DU145, intermediate in LNCaP, and highest in abl (FIG. 14F). FIG. 14F is a heat map demonstrating the transcript levels of EZH2-activated genes that are functionally involved in either base excision repair or mismatch repair pathway among LNCaP, abl, and DU145. Taken together, these findings revealed a close connection between the tumor-suppressive activity of EZH2 inhibitors and DNA repair machinery, which suggests a new indication for these compounds as anticancer drugs.

### Example 6-EZH2 Inhibitors Enhance Responses of Prostate Cancer Cells to DNA Damaging Agents

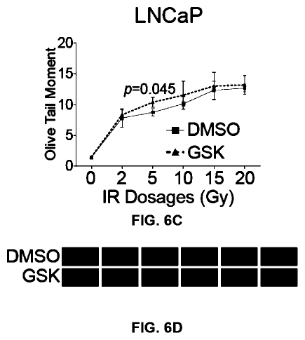

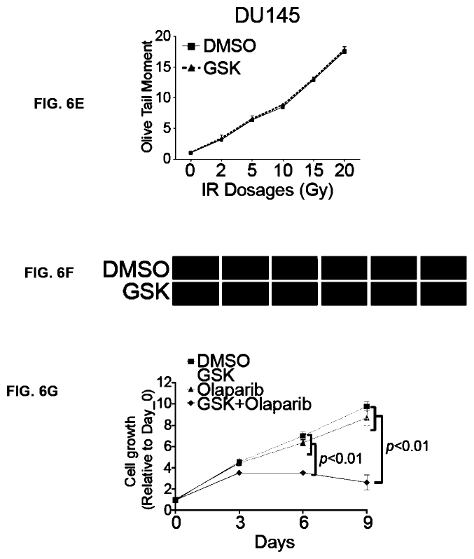

EZH2i can be administered to sensitize CRPC cells to DNA damage repair (DDR) agents. To validate this, abl, LNCaP and DU145 cells were exposed to increasing doses of ionizing radiation (IR). While abl cells became much more sensitive to IR after GSK126 treatment (FIGS. 6A-6B), this difference was very minor in LNCaP (FIGS. 6C-6D) and non-existent in DU145 (FIG. 6E-6F). FIG. 6A is a graphical representation of the alkaline comet assays in abl cells. Prostate cancer cells were pretreated with vehicle (DMSO) or 5 μM GSK126 (GSK) for 7 days, and then exposed to increasing dosages of ionizing radiation (IR) followed by recovery for 4 hrs. FIG. 6B is a set of photographic images of each IR dosage corresponding to the dosages in FIG. 6A. FIG. 6C is a graphical representation of the alkaline comet assays in LNCaP cells. Prostate cancer cells were pretreated with vehicle (DMSO) or 5 μM GSK126 (GSK) for 7 days, and then exposed to increasing dosages of ionizing radiation (IR) followed by recovery for 4 hrs. FIG. 6D is a set of photographic images of each IR dosage corresponding to the dosages in FIG. 6C. FIG. 6E is a graphical representation of the alkaline comet assays in DU145 cells. Prostate cancer cells were pretreated with vehicle (DMSO) or 5 μM GSK126 (GSK) for 7 days, and then exposed to increasing dosages of ionizing radiation (IR) followed by recovery for 4 hrs. FIG. 6F is a set of photographic images of each IR dosage corresponding to the dosages in FIG. 6D.

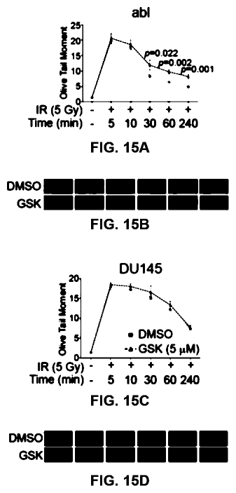

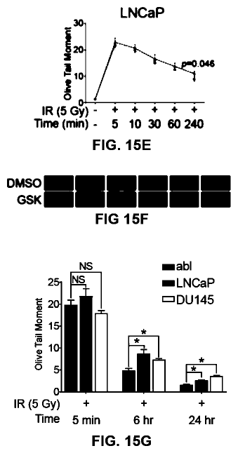

In addition, when 5 gray (Gy) of IR was applied and cell recovery was monitored, drastically delayed DNA damage repair was observed in abl cells pretreated with GSK126 (FIGS. 15A-15B), but not in LNCaP (FIGS. 15C-15D) nor in DU145 (FIGS. 15E-15F). FIG. 15A is a graphical representation of the alkaline comet assay results in abl cells after pretreatment with vehicle (DMSO) or 5 μM GSK126 (GSK) for 7 days and then being exposed to 5 gray (Gy) ionizing radiation (IR) followed by recovery at indicated time points (Quantification of the results using “Olive Tail Moment” parameter). FIG. 15B is a set of representative images of each time point corresponding the sampling in FIG. 15A. FIG. 15C is a graphical representation of the alkaline comet assay results in LNCaP cells after pretreatment with vehicle (DMSO) or 5 μM GSK126 (GSK) for 7 days and then being exposed to 5 gray (Gy) ionizing radiation (IR) followed by recovery at indicated time points (Quantification of the results using “Olive Tail Moment” parameter). FIG. 15D is a set of representative images of each time point corresponding the sampling in FIG. 15C. FIG. 15E is a graphical representation of the alkaline comet assay results in DU145 cells after pretreatment with vehicle (DMSO) or 5 μM GSK126 (GSK) for 7 days and then being exposed to 5 gray (Gy) ionizing radiation (IR) followed by recovery at indicated time points (Quantification of the results using “Olive Tail Moment” parameter). FIG. 15F is a set of representative images of each time point corresponding the sampling in FIG. 15E. Without EZH2i pretreatment, IR has moderate effects in abl even at a dose as high as 20 Gy (FIG. 15G). FIG. 15G is box plot showing the olive tail moment values in abl, LNCaP and DU145 cell after being treated with 5 gray (Gy) ionizing radiation (IR) followed by recovery at indicated time points. This indicates that abl may represent a radioresistant scenario and EZH2 inhibitors may be considered to overcome radiotherapy resistance in advanced prostate cancer.

PARP-1 is an ADP-ribosylating enzyme involved in various forms of DNA repair, including BER. It has been reported that deficiencies of any components in BER pathway resulted in hypersensitivity of cancer cells to PARP inhibitors. The biological effect of combining EZH2i with an PARP-1 inhibitor, such as olaparib, on proliferation of abl cells was investigated (FIG. 6G). FIG. 6G is a graphical representation of the combination therapy of EZH2 inhibitor and olaparib to abrogate the androgen-independent growth of abl cells. Cells were treated with vehicle (DMSO), 0.5 μM GSK126 (GSK) alone, 1 μM Olaparib alone, or both drugs (GSK+Olaparib) for indicated days, and cell proliferation were determined using Cell Counting Kit-8. Combined treatment of EZH2i and olaparib greatly suppressed abl cell growth compared to each drug alone. Taken together, a novel therapeutic strategy for hormone independent prostate cancer was developed to exploit the suppressive effects of EZH2 inhibitors on DNA damage repair to sensitize cancer cells to DNA damaging agents.

### Example 7—The Expression of DNA Damage Repair Genes Predicts Sensitivity of Cancer Cells to EZH2 Inhibitors

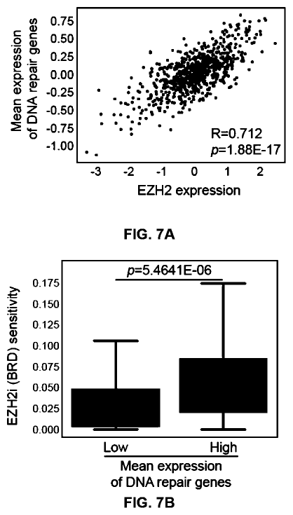

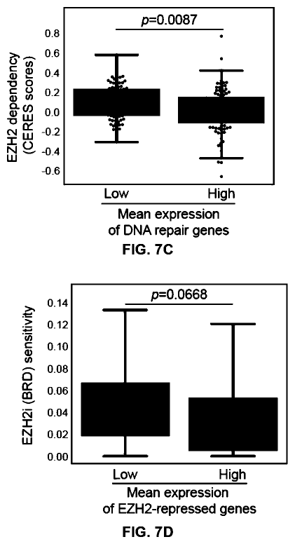

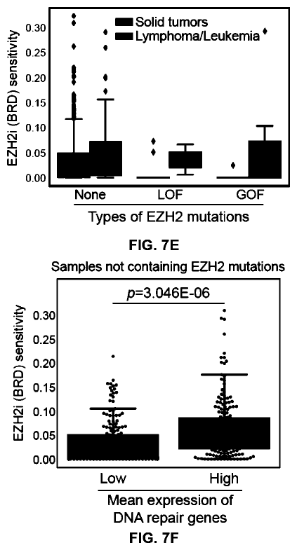

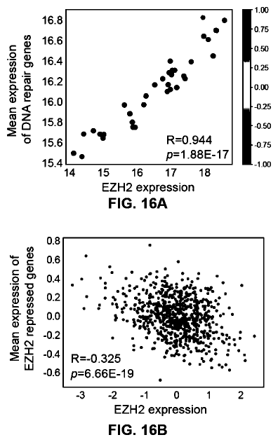

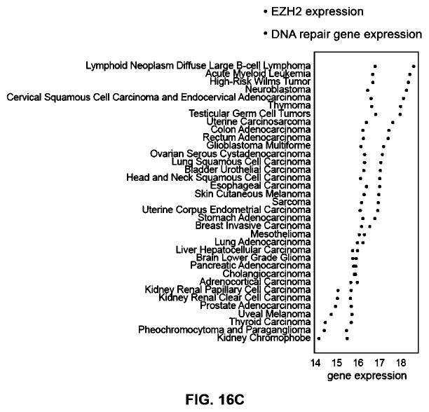

To examine whether the treatment regimen in prostate cancer cells can be generalized to other types of cancers, the expression correlation between EZH2 and the DNA damage repair genes were evaluated in the Cancer Cell Line Encyclopedia (CCLE) dataset. Across various cancer cell lines, expressions between DNA damage repair genes and EZH2 were strongly correlated. FIG. 7A is a graphical representation of the expression correlation between EZH2 and DNA damage repair genes in cancer cells from CCLE gene expression data. Each dot represents one cell line. All clinical samples from TCGA data sets confirmed the expression profile, and within individual cancer types, there were strong positive expression correlations. FIG. 16A is a graph showing expression correlation between EZH2 and DNA damage repair genes within one particular type of cancers from TCGA data. Each dot represents one cancer type; color scale, correlation coefficient of all samples within individual cancer type. These results indicate that EZH2-mediated control of DNA damage repair machinery represents a target for development of treatment regimens. To examine whether expression of DNA damage repair genes can dictate EZH2i sensitivities, Cancer Therapeutics Response Portal (CTRP) compound screen data was analyzed to measure sensitivities to EZH2 inhibitor BRD-K62801835-001-01-0 (BRD) in 668 cancer cells lines of various types. Expressions of DNA repair genes were positively associated with cell sensitivities to BRD, as the top 25% cell lines with the highest levels of these genes were much more susceptible to BRD treatment than the bottom 25% cells with the lowest expression. FIG. 7B is a box plot showing the sensitivity of CTRP cell lines to EZH2 inhibitor (BRD, BRD-K62801835-001-01-0). Cells were grouped based on their mean expression of DNA damage repair genes, and then sensitivities to BRD in the top 25% cells with the highest expression were compared with those in bottom 25% cells with the lowest expression.

The expression of DNA repair genes and EZH2 dependency was further confirmed using the genome-wide CRISPR-Cas9 knockout screen data across 389 cancer cell lines. The higher the expression of DNA repair genes, the more cells depend on EZH2 for sustained growth, which was reflected by lower CERES scores of EZH2. FIG. 7C is a box plot illustrating the correlation between expression of DNA damage repair genes and dependency of EZH2. EZH2 dependency scores (CERES scores) of all the cell lines were retrieved from the DepMap CRISPR-Cas9 knockout screening data. In contrast, genes that were repressed by EZH2, although having negative expression correlation with EZH2 (FIG. 16B), do not consistently predict EZH2i sensitivity in cancer cells (FIG. 7D). FIG. 16B is a scatter plot illustrating the expression correlation between EZH2 and EZH2-repressed genes from CCLE data. FIG. 7D is a box plot showing the sensitivity of CTRP cell lines to EZH2 inhibitor (BRD, BRD-K62801835-001-01-0). Cells were grouped based on their mean expression of EZH2-repressed genes, and then sensitivities to BRD in the top 25% cells with the highest expression were compared with those in bottom 25% cells with the lowest expression.

Notably, the top two cancer types with highest expressions of EZH2 and DNA repair genes are diffuse large B-cell lymphoma (DLBCL) and acute myeloid leukemia (AML). FIG. 16C is a dot plot representing expression levels of EZH2 (red dots) and DNA damage repair genes (blue dots) in each specified cancer type in TCGA data. As these hematopoietic malignancies harbor EZH2 mutant forms that claimed to affect its methyltransferase activity and therefore orchestrate the sensitivities of cancer cells to EZH2 inhibitors, their prevalent presence in these analyses raised the concern that the observed biomarker profile was actually due to EZH2 mutations. Two pieces of evidence indicated the biomarker profile was not due to EZH2 mutations. First, no significant difference in EZH2i-senstivity was observed when the cancer cells were grouped according to EZH2 mutation status. FIG. 7E is a box plot showing the sensitivity to EZH2 inhibitor (BRD, BRD-K62801835-001-01-0) in three different groups of cancer cells based on the statuses of EZH2 mutations. All the lymphoma and leukemia cells were first separated from solid tumors, with further classification according to the presence of EZH2 somatic mutations. GOF, gain-of-function mutations at residues Y641, A677 or A687; LOF, loss-of-function mutations; None, no mutations. EZH2i does not show higher potency in cells containing gain-of-function (GOF) mutations of EZH2 compared to those with loss-of-function (LOF) mutations or no mutations (GOF vs. LOF, p=0.2705; GOF vs. None, p=1.000). Second, the correlation between DNA repair genes and EZH2i-responsiveness persisted even after excluding tumors with EZH2 mutations. FIG. 7F is box plot illustrating the correlation between the expression of DNA damage repair genes and sensitivity of cancer cells to EZH2 inhibitor (BRD, BRD-K62801835-001-01-0). Cells containing EZH2 somatic mutations were excluded from the analysis. Therefore, a group of DNA damage repair genes are activated by EZH2, and evaluation of the levels of expression of these genes can help one assess cancer cell responsiveness to EZH2 inhibition.

To provide a more concise description, some of the quantitative expressions herein are recited as a range from about amount X to about amount Y. It is understood that wherein a range is recited, the range is not limited to the recited upper and lower bounds, but rather includes the full range from about amount X through about amount Y, or any amount or range therein. To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

### Example 8—Methods

Antibodies and Reagents. Antibodies used in this study include: αAR (H-280, sc-13062) for ChIP-qPCR and ChIP-Seq; αAR (N-20, sc-816) for Western blot and immunoprecipitation; αH3K27me3 (C36B11, #9733S) for ChIP-qPCR, ChIP-Rx and Western blot; αH2Av (39715) for ChIP-Rx; αAR (441, sc-7305), αH3K27me1 (ab194688), αH3K27me2 (ab24684), αH3 (ab4086), αEZH2 (clone 11, 612666), αCDCA3 (FL-268, sc-134625), αKIAA0101 (SAB1406878), αTACC3 (C-2, sc-376883), αHA (51064-2-AP), αBIRC5 (D-8, sc-17779), αCKS2 (F-12, sc-376663), αMUTYH (C-6, sc-374571), αDNA pol ε A (34, sc-135885) for immunoblotting. EZH2 inhibitors were purchased from Xcessbio Biosciences Inc. (GSK126, M60071 and EPZ-6438, M60122) and olaparib (AZD2281) from Selleck Chemicals (S1060). The SMARTpool siRNAs (Dharmacon) used in this study were: siGENOME Non-Targeting siRNA Pool #2 (D-001206-14), SMARTpool ON-TARGETplus EZH2 siRNA (L-004218-00) and SMARTpool siGENOME EZH2 siRNA (M-004218-03).

Normal and Cancer Prostate Epithelial Cell Lines and Culture Conditions. Benign and malignant prostatic epithelial cell lines RWPE-1, DU145, PC3, and LNCaP were originally purchased from the American Type Culture Collection. LHSAR cell line was kindly provided by Dr. Matthew Freedman. LAPC4, LNCaP-AI and LAPC4-CR were all obtained from Dr. Philip W. Kantoff's lab. LNCaP-abl (abl) cell line was generously shared by Zoran Culig (Innsbruck Medical University, Austria). VCaP and CWR22Rv1 cell lines were graciously provided by Dr. Steven P. Balk. C4-2B was obtained from ViroMed Laboratories (Minneapolis, Minn.). All of these cell lines were authenticated at Bio-Synthesis Inc. and confirmed to be mycoplasma-free using MycoAlert Mycoplasma Detection Kit (Lonza). The specific culture conditions for each cell line were listed in Table 1.

TABLE 1
Prostate cell lines and their culture conditions.

| | | | |
|---|---|---|---|
| | | | Culture |
| Cell Names | Culture Medium | Supplements | Condition |

Prostate epithelial cell lines

| | | | |
|---|---|---|---|
| LHSAR | PrEBM basal | PrEGM SingleQuot Kit Suppl. & Growth | 37° C., 5% CO2 |
| | medium | Factors | |
| RWPE-1 | Keratinoyte serum | 0.05 mg/mL bovine pituitary extract | 37° C., 5% CO2 |
| | free medium | (BPE), 5 ng/mL human recombinant | |
| | | epidermal growth factors (EGF) | |

AR-null prostate cancer cell lines

| | | | |
|---|---|---|---|
| DU145 | phenol-red-free | 10% charcoal-stripped FBS, 1% | 37° C., 5% CO2 |
| | RPMI1640 | Penicillin-Streptomycin | |
| PC3 | regular DMEM | 10% FBS, 1% Penicillin-Streptomycin | 37° C., 5% CO2 |

AR-positive, androgen-dependent prostate cancer cell lines

| | | | |
|---|---|---|---|
| LAPC4 | regular RPMI1640 | 10% FBS, 1% Penicillin-Streptomycin | 37° C., 5% CO2 |
| LNCaP | regular RPMI1640 | 10% FBS, 1% Penicillin-Streptomycin | 37° C., 5% CO2 |
| VCaP | regular DMEM | 15% FBS, 1% Penicillin-Streptomycin, | 37° C., 5% CO2 |
| | | 1% Non-essential amino acids | |

AR-positive, androgen-independent prostate cancer cell lines

| | | | |
|---|---|---|---|
| C4-2B | regular RPMI1640 | 10% FBS, 1% Penicillin-Streptomycin | 37° C., 5% CO2 |
| CWR22Rv1 | regular DMEM | 10% FBS, 1% Penicillin-Streptomycin | 37° C., 5% CO2 |
| LAPC4-CR | phenol-red-free | 10% charcoal-stripped FBS, 1% | 37° C., 5% CO2 |
| | RPMI1640 | Penicillin-Streptomycin | |
| LNCaP-abl | phenol-red-free | 10% charcoal-stripped heat-inactivated | 37° C., 5% CO2 |
| | RPMI1640 | FBS, 1% Penicillin-Streptomycin | |
| LNCaP-AI | phenol-red-free | 10% charcoal-stripped FBS, 1% | 37° C., 5% CO2 |
| | RPMI1640 | Penicillin-Streptomycin | |

Cell Proliferation Assay. Normal prostate epithelial cells and prostate cancer cells were seeded at optimal density in 384-well plates using an automated dispensing system (BioTek EL406). EZH2 inhibitors (GSK126 or EPZ-6438) were subjected to a 10-point series of threefold dilution (from 0.632 nM to 20 μM) in DMSO and then added into cells by robotic pin transfer in a JANUS workstation. Each drug at a certain dose in every specific cell line had four replicates. After 7 days of incubation, cellular ATP levels were measured using ATPlite Luminescence Assay (PerkinElmer). Data were normalized to the number of cells under DMSO conditions, and IC50 were determined with GraphPad Prism software.

Standard ChIP and ChIP-Seq assays. Chromatin immunoprecipitation (ChIP) experiments were performed as previously described. Basically, cells were crosslinked with 1% formaldehyde and lysed in RIPA buffer with 0.3 M NaCl. ChIP DNA was purified using PCR Purification Kit (Qiagen) and then quantified by Quant-iT™ dsDNA HS Assay Kit (Invitrogen). Equal amounts of ChIP enriched DNA (5-10 ng) under each treatment condition (DMSO, GSK126 or EPZ-6438) were prepared for either targeted ChIP-qPCR or ChIP-Seq libraries. For protein detection in ChIP samples, SDS sample buffer was added to the reverse crosslinked input lysates, which were then subjected to Western blot analysis. ThruPLEX-FD Prep Kit (Rubicon Genomics) was used to construct the sequencing libraries according to the manufacturer's protocol, and the final products were sequenced on the NextSeq 500. For targeted ChIP-qPCR, purified ChIP DNA was subjected to real-time quantitative PCR with specific primers as listed in Table 2.

TABLE 2
Primers for targeted ChIP-qPCR. Table 2 discloses
SEQ ID NOS 1-28, respectively, in order of
appearance.

| Names | Sequences |
|---|---|
| KIAA0066 F | CTAGGAGGGTGGAGGTAGGG[4] |
| KIAA0066 R | GCCCCAAACAGGAGTAATGA[4] |
| PPIA F | GCCAGGCTCCTGTTTTAATG |
| PPIA R | GCAGTCTCCGGTTTTGAGAG |
| CCND2 F | TCCAACCGAAACTCCAAAAC[5] |
| CCND2 R | CTTTTCACCCTTCACGGAAA[5] |
| DAB2IP F | CCTGCTCTGAGTCTGCACTG[5] |
| DAB2IP R | TCGAATCTCTCCCATGGTTC[5] |
| p16 F | AGGGGAAGGAGAGAGCAGTC[6] |
| p16 R | GGGTGTTTGGTGTCATAGGG[6] |
| SHH F | TCCTTCCATTTCCACTCCTG |
| SHH R | TCTTGCTACAATGGCCTTCC |
| TNFSF9 F | GCGATTTCTTGGCGTTACTT |
| TNFSF9 R | TCGGGGAGGTTAGAGTGCT |
| VIM F | CAATCTCAGGCGCTCTTTGT |
| VIM R | GAGCGGGAAGAGGAAAGAGTA |
| ETV4 F | TCTCCAGCCTATGCACTCCT |
| ETV4 R | CTTCCATTTGCACAAGCAGA |
| FOXC1 F | CCCTCTCTTGCCTTCTTCCT |
| FOXC1 R | CGTCAGGTTTTGGGAACACT |
| GATAS F | CCCTAACTGGGAAAACACGA |
| GATAS R | CGCCCAGGTAAATCCAAGTA |
| HOXD8 F | AATAGTTCGGGTGCGTTTTG |
| HOXD8 R | TCACTGGCCCAATCTTTTTC |
| NFkB2 F | GGGGTGGGGAAGTAATAGGA |
| NFkB2 R | CCTTAGCAGGTGCCATGAGT |
| RUNX3 F | CATGGACCGTAGTCTTTTCT |
| RUNX3 R | CACTGCCAAGAACGCACTTA |

ChIP-Rx of H3K27me3 in Prostate Cancer Cells. To quantitatively measure the changes of H3K27me3 abundance upon EZH2 inhibitor treatments, H3K27me3 ChIP with reference exogenous genome (ChIP-Rx) was performed as described. Briefly, 5 μg of ready-to-ChIP human chromatin from abl or DU145 cells were mixed with 125 ng of Drosophila chromatin that has been sheared to proper sizes. 4 uL of H3K27me3 antibody together with 0.2 uL of Drosophila-specific H2Av antibody were added to the mixture. Each sample was then treated as one and subjected to standard processes of ChIP and sequencing. Short reads obtained from the sequencer were mapped to human genome (hg19) and drosophila genome (dm3) respectively, and peaks were called using MACS v2.0. Overall, 12,114 and 131,469 peaks of H3K27me3 were identified in abl and DU145 cells under DMSO treatment condition based on FDR<0.01. Normalization ratios between treatment and control groups were calculated based on the H2Av read counts mapped to drosophila genome between EPZ-6438 and DMSO or GSK126 and DMSO. The enrichment changes of human H3K27me3 signals were then normalized by the corresponding normalization ratios.

Cell Transfections. A total of 50 pmol (for each well in 24-well plate) or 100 pmol (for each well in 6-well plate) of each siRNA was transfected into abl or DU145 cells using Lipofectamine RNAiMAX reagent (Invitrogen) according to the manufacturer's instructions. Cell from 24-well plates were collected at indicated time points and counted after Trypan Blue staining for cell numbers, or harvested 48 hrs after transfection for RNA extraction, or lysed 72 hrs post-transfection and subjected to Western blot.

RNA isolation and RT-qPCR. RNA was extracted and purified using the TRIzol Reagent combined with RNeasy Mini Kit (Qiagen) according to manufacturer's protocols. 2 μg of total RNAs were then used for cDNA synthesis using High Capacity cDNA Reverse Transcription Kit (Applied Biosystems). Real-time quantitative RT-PCR was performed, and gene expression was calculated as described previously, using the formula 2-ΔΔCt relative to the level of GAPDH as the internal control. Sequences of RT-qPCR primers were listed in Table 3.

TABLE 3
Primers for quantitative real-time RT-qPCR

| | | SEQ |
|---|---|---|
| | | ID |
| Gene Names | Sequences | NO: |
| TACC3 mRNA F | GCACAGGATTCTAAGTCCTAGCA | 29 |
| TACC3 mRNA R | CCAGACCGGGTGTGAGTTTT | 30 |
| KIAA0101 mRNA F | ATGGTGCGGACTAAAGCAGAC | 31 |
| KIAA0101 mRNA R | CCTCGATGAAACTGATGTCGAAT | 32 |
| BIRC5 mRNA F | AGGACCACCGCATCTCTACAT | 33 |
| BIRC5 mRNA R | AAGTCTGGCTCGTTCTCAGTG | 34 |
| CDCA3 mRNA F | CTGGAGGGTCTTAAACATGCC | 35 |
| CDCA3 mRNA R | CACTGCTGGTCTTCATAGGTG | 36 |
| SHH mRNA F | CCAAGGCACATATCCACTGCT | 37 |
| SHH mRNA R | GTCTCGATCACGTAGAAGACCT | 38 |
| VIM mRNA F | AGTCCACTGAGTACCGGAGAC | 39 |
| VIM mRNA R | CATTTCACGCATCTGGCGTTC | 40 |
| MAT1A mRNA F | ATCAGGGTTTGATGTTCGGCT | 41 |
| MAT1A mRNA R | GCGTTGAGCTTGTGAGCAA | 42 |
| TNFSF9 mRNA F | GGCTGGAGTCTACTATGTCTTCT | 43 |
| TNFSF9 mRNA R | ACCTCGGTGAAGGGAGTCC | 44 |
| CKS2 mRNA F | TTCGACGAACACTACGAGTACC | 45 |
| CKS2 mRNA R | GGACACCAAGTCTCCTCCAC | 46 |
| APEX1 mRNA F | GTTTCTTACGGCATAGGCGAT | 47 |
| APEX1 mRNA R | CACAAACGAGTCAAATTCAGCC | 48 |
| POLE mRNA F | TTCCTCAGTTTCGGCACTCAA | 49 |
| POLE mRNA R | CTCAAAACCAAACCGCAAATCC | 50 |
| POLE3 mRNA F | GCTGTACGCCACATCCTGT | 51 |
| POLE3 mRNA R | TTCAATGGGGTAACGAACCGC | 52 |
| MUTYH mRNA F | TGCCACGTACAGCAGAGAC | 53 |
| MUTYH mRNA R | CAAAGGCGATAGAGGCAATGG | 54 |
| FOXC1 mRNA F | TGTTCGAGTCACAGAGGATCG | 55 |
| FOXC1 mRNA R | ACAGTCGTAGACGAAAGCTCC | 56 |
| ETV4 mRNA F | GCAACGGAATTTCCTGAGATCC | 57 |
| ETV4 mRNA R | ACGGAGCTATGTTCCCCGA | 58 |
| GATA6 mRNA F | GTGCCAACTGTCACACCACA | 59 |
| GATA6 mRNA R | GAGTCCACAAGCATTGCACAC | 60 |
| HOXD8 mRNA F | GGAAGACAAACCTACAGTCGC | 61 |
| HOXD8 mRNA R | TCCTGGTCAGATAGGGGTTAAAA | 62 |
| RUNX3 mRNA F | AGCACCACAAGCCACTTCAG | 63 |
| RUNX3 mRNA R | GGGAAGGAGCGGTCAAACTG | 64 |
| NFkB2 mRNA F | AGAGGCTTCCGATTTCGATATGG | 65 |
| NFkB2 mRNA R | GGATAGGTCTTTCGGCCCTTC | 66 |
| KCNMB4 mRNA F | AGTGCTCCTATATCCCTCCCT | 67 |
| KCNMB4 mRNA R | GCTGGGAACCAATCTCATCTTT | 68 |
| GAPDH mRNA F | CGAGATCCCTCCAAAATCAA | 69 |
| GAPDH mRNA R | TTCACACCCATGACGAACAT | 70 |

Cell Cycle Analysis with Flow Cytometry. Prostate cancer cells were pre-treated with nocodazole (5 μg/mL) for 24 hrs, and were released by being replenished with fresh medium. Cells were then incubated with GSK126 or EPZ-6438 at final concentrations of 5 μM for days as indicated. Cell cycle analyses were performed using previously published protocols. Generally, cells were collected, washed with ice-cold PBS, and fixed in 70% ethanol for at least 1 hr on ice. Cells were then pelleted, washed with PBS, and incubated in propidium iodide solution (Sigma, P4864) with RNase A (Sigma, R6513) for 30 min at 37° C. Flow cytometry analyses were done using an LSRII flow cytometer (Becton Dickinson).

Comet Assay. Prostate cancer cells were treated under described conditions and the Alkaline Comet Assay was then performed following the manufacturer's instructions (Trevigen, #4250-050-K). Briefly, treated or untreated cells were harvested and resuspended in ice cold PBS (Ca2+- and Mg2+-free) at a density of 1×105 cells/ml, mixed with molten LMAgarose (1:10 ratio) and 50 μl of the mixture was immediately pipetted onto Comet Slide. After the agarose was solidified at 4° C. in the dark for 15 minutes, the slides were immersed first in Lysis Solution for 1 hr and then in Alkaline Unwinding Solution (200 mM NaOH, 1 mM EDTA, pH>13) for another 1 hr at 4° C. in the dark. Alkaline electrophoresis was conducted at 4° C. for 30 minutes at 21 volts. Cells were then fixed with 70% ethanol and stained with SYBR Gold. Comet images were taken by Nikon Ni-U fluorescence microscopy (Nikon) using a FITC filter. Comet tail moments were assessed using CometScore.v2.0 (TriTek Corp., Sumerduck, Va. 22742, USA). Olive tail moment is defined as Tail DNA % x Tail Moment Length that is measured from the center of the head to the center of the tail. The quantification of Olive tail moments from each condition was calculated from a minimum of 100 cells for each data point.

Test of EZH2 Inhibitors in Xenograft Mouse Model. Male nude mice (Taconic) were castrated after 3 days of accommodation. CWR22Rv1 xenografts were established in the flanks of mice by injecting ˜2 million cells in 50% Matrigel (BD Biosciences). When tumors reached approximately 200 mm³, mice started to receive daily injection of EZH2 inhibitors (provided by Xcessbio Biosciences Inc) dissolved in 20% captisol (CYDEX, NC-04A-120106). Tumors were measured 3 times every week and harvested after 3 weeks. Frozen samples were subjected to RT-qPCR and immunoblotting to study the expression of selected genes. All animal protocols were approved by the Beth Israel Deaconess Institutional Animal Care and Use Committee, and the experiments were performed in accordance with institutional and national guidelines.

Data Collection. EZH2 ChIP-Seq data and EZH2 siRNA microarray expression data were both retrieved from our previous study (GSE39461). The Cancer Therapeutics Response Portal (CTRP) compound screen data was used in FIG. 7A in order to correlate gene expression with cell sensitivity to EZH2 inhibitors. Two independent prostate cancer cohorts were retrieved and analyzed in FIGS. 5F and G and FIGS. 14D and 14E. In FIG. 14F, the LNCaP RNA-Seq data sets were downloaded from the published data sets.

Analysis of EZH2 Inhibitor Mediated Gene Expression by RNA-Seq. Both abl and DU145 cells were treated with EZH2 inhibitors (GSK126 or EPZ-6438) at final concentrations of 5 μM for 60-72 hrs before RNAs were extracted. RNA-seq library was prepared using Illumina True-seq RNA sample preparation kit and sequenced to 50 bp using Illumina Hi-seq platform. RNA-seq data was mapped to human genome (hg19) using TopHat version 2.0.6. DESeq2 was applied to calculate the logarithmic fold change (LFC) and p-value in order to call any significantly changed genes between treatment and control groups. Differentially expressed genes were first filtered using LFC>0.5 or <−0.5, and then top 200 genes were selected through ranking by their p-values. The authentic target genes of EZH2 in abl cells were defined as those showing similar expression changes upon either EZH2 silencing or inhibitor treatment.

Focused CRISPR screen library design. To design a smaller scale CRISPR/Cas9 knockout screen library focusing on cancer-related genes, we selected 6000 genes based on the reported literatures. For each gene, we designed ten single-guide RNAs (sgRNAs) with 19 bp against its coding region with optimized cutting efficiency and minimized off-target potentials. Cutting efficiency wise, we used sequence features of the spacers to calculate the efficiency score for each sgRNA using predictive model. Off-target wise, we used BOWTIE to map all candidate sgRNAs to hg38 reference genome, and chose those with least potential off-targets. We selected the 10 best sgRNAs for each gene based on the criteria above. The library also contains positive and two types of negative controls (non-targeting controls and non-essential regions-targeting sgRNAs). Positive controls: we included 1466 sgRNAs targeting 147 positive control genes, which are significantly negatively selected in multiple screen conditions. Non-targeting negative controls: 795 sgRNAs with sequences not found in genome. Non-essential regions-targeting negative controls: 1891 sgRNAs targeting AAVS1, ROSA26, and CCR5, which have been reported as safe-harbor regions where knock-in leads to few detectable phenotypic and genotypic changes.

Plasmid construction and lentivirus production. The sgRNA library was synthesized at CustomArray© and then amplified by PCR. The PCR products were subsequently ligated into lentiCRISPR V2 plasmid, followed by transformation into competent cells according to an online protocol (GeCKO library Amplification Protocol from Addgene). Afterwards, we isolated the plasmid and constructed a sequencing library for Miseq to ensure library diversity. To make lentivirus, T-225 flasks of 293FT cells were cultured at 40%˜50% confluence the day before transfection. Transfection was performed using X-tremeGENE HP DNA Transfection Reagent (Roche). For each flask, 20 μg of lentivectors, 5 μg of pMD2.G, and 15 μg of psPAX2 (Addgene) were added into 3 ml OptiMEM (Life Technologies). 100 ul of X-tremeGENE HP DNA Transfection Reagent was diluted in 3 ml OptiMEM and, after 10 min, it was added to the plasmid mixture. The complete mixture was incubated for 20 min before being added to cells. After 6 hr, the media was changed to 30 ml DMEM+10% FBS. The media was removed 60 hrs later and centrifuged at 3,000 rpm at 4° C. for 10 min to pellet cell debris. The supernatant was filtered through a 0.45 μm membrane with low protein binding. The virus was ultracentrifuged at 24,000 rpm for 2 hr at 4° C. and then resuspended overnight at 4° C. in DMEM+10% FBS. Aliquots were stored at −80° C.

CRISPR screens. LNCaP or abl were kept in their respective medium that is routinely used to maintain normal proliferation. Cells of interest were infected at a low MOI (0.3˜0.5) to ensure that most cells receive only 1 viral construct with high probability. Briefly, 3×106 cells per well were plated into a 12 well plate in the appropriate standard media supplemented with 8 μg/ml polybrene. Each well received a different titrated virus amount, usually between 5 and 50 ul, along with a non-transduction control. The 12-well plate was centrifuged at 2,000 rpm for 2 hr at 37° C. After the spin, media was aspirated and fresh media without polybrene was added. Cells were incubated overnight and then enzymatically detached using trypsin. Cells were counted and each well was split into duplicate wells. Each replicate of LNCaP or abl cells received 4 μg/mL puromycin. After 3 days or as soon as no surviving cells remained in the non-transduction control under puromycin selection, cells were counted. Percent transduction was calculated as cell numbers from the replicate with puromycin divided by cell counts from the replicate without puromycin and then multiplied by 100. The virus volume yielding a MOI closest to 0.4 was chosen for large-scale screening. For focused CRISPR-Cas9 knockout screen, large-scale spin-infection of 2×108 cells was carried out using four of 12-well plates with 4×106 cells per well. Wells were pooled together into larger flasks on the same day after spin-infection. After three days of puromycin selection, the surviving abl cells were divided into three groups: one for day 0 control, and the other two cultured in the presence of DMSO or GSK126 for four weeks. Two rounds of PCR were performed after gDNA had been extracted, and 300 μg DNA per sample was used for library construction. Each library was sequenced at 3˜30 million reads to achieve ˜300× average coverage. The day 0 sample library served as the control to identify genes or pathways that were positively or negatively selected.

CRISPR screen normalization and analysis. CRISPR knockout screen data were analyzed following MAGeCK protocol, which assigns each gene with a beta score (β), analogy of log fold change. Positive and negative beta scores indicate positive and negative selection, respectively. Considering that cells in screens may be harvested at different time points, normalization of screen duration was carried out to ensure comparable CRISPR screens. Equation of cell growth is equivalent to that of beta score defined in MAGeCK:

Cₜ=C₀·e^{r}^{ᵢ}^{t}↔Cₜ=C₀·e^{β}^{ᵢ}

Where rᵢ is growth constant of cells with gene gᵢ knockout, and t is the duration of screen. The equivalence of these two equations shows that beta scores are linearly dependent on screen duration, suggesting the necessity of equalizing the screen durations for fair screen comparisons. Considering that the pan-essential genes are negatively selected similarly in different conditions, the absolute median beta score of the pan-essential genes is proportional to the screen duration. Assuming the r of essential genes remains constant in various screen conditions, then:

β_{essential genes}∝t

Therefore, we can rescale the beta scores using the absolute median beta scores of known-essential genes:

$${β}_{i - normalize} = {r}_{i} = \frac{{β}_{i}}{t} ∝ \frac{{β}_{i}}{\vert median ( {β}_{e s s e ntial genes} ) \vert}$$

The methyltransferase EZH2 has been a focus of cancer drug development for several years. Inhibitors of EZH2 have been tested in patients with non-Hodgkin lymphoma or solid tumors harboring the gain-of-function (GOF) mutations of EZH2 or genetic aberrations in components of SWI/SNF complex (INI1 and SMARCA4), respectively. The therapeutic strategy was based on the overall reduction of H3K27me3 levels upon EZH2i treatment in these cases and that cancer cell lines harboring these mutations were especially sensitive to the compounds. In contrast, this disclosure provides for a gene signature, which is functionally involved in DNA damage repair that allows for assessment of responses of cancer cells to EZH2i rather than the mutational status of EZH2. Using CRISPR-based knockout screens, certain DNA damage repair genes, particularly those in BER pathway, were identified as required for the inhibitory effects of EZH2i in CRPC cells. The expression of these genes was acutely and robustly downregulated upon pharmacological inhibition of EZH2, and their levels are highly correlated with cellular sensitivities to EZH2 inhibitors. In lymphomas, such as follicular lymphoma (FL), cases with activating somatic mutations of EZH2 are generally more susceptible to EZH2 inhibitors, and presence of these genetic alterations is indicative of EZH2i efficacy. In the contrary, despite of repeatedly implication of EZH2 involvement in driving aggressive phenotypes in solid tumors, the lack of EZH2 mutations often raises concerns in conducting trials of EZH2 inhibitors in these types of cancers. Here, EZH2 inhibitors were used in wild-type EZH2 expressing cancers, such as the metastatic, hormone-refractory prostate cancer. This effectiveness is positively associated with the expression of DNA damage repair genes. The gene signature reliably predicted sensitivities of a variety of cancer cells to EZH2 inhibitors. Therefore, EZH2 inhibitors can be part of an effective therapeutic regimen, and expression profile of certain DNA damage repair genes was an accurate biomarker in predicting the drug sensitivities in cancers that do not carry EZH2 somatic mutations.

A new combination therapy strategy was developed to leverage the crosstalk between EZH2 and DNA damage repair machinery.

Pretreatment with EZH2 inhibitors significantly boosted the tumor-suppressive effects of DNA damaging agents, such as ionizing radiation and olaparib. Thus, combination of EZH2 inhibitors and genotoxic agents is an alternative approach in anticancer therapy. Embodiments include a method for sensitizing cancer cells in a subject in need of chemotherapy by administering to the subject a therapeutically effective amount of an Enhancer of Zeste Homolog 2 (EZH2) inhibitor before administration of a poly (ADP-ribose) polymerase 1 inhibitor. These cancer cells in the subject are characterized by elevated levels of expression of one or more of Cell Division Cycle-Associated Protein 3, CDC28 Protein Kinase Regulatory Subunit 2, MutY DNA Glycosylase, DNA Polymerase Epsilon 3, and Transforming Acidic Coiled-Coil Containing Protein 3 as compared to normal cells. Based on the data here in CRPC cell model and analysis across hundreds of cancer cell lines, EZH2 inhibitors can be administered to overcome radiotherapy resistance in advanced cancer and improve the efficacy of olaparib in BRCA-deficient or even BRCA-proficient tumors. A combination of DNA damaging therapeutics with EZH2 inhibitors can be the treatment regimen for some hard-to-treat cancer types. One such regimen can include administering pharmaceutical compositions containing an EZH2 inhibitor and a PARP-1 inhibitor.

Those skilled in the art will recognize, or be able to ascertain using no more than routine experimentation, many equivalents to the specific embodiments of the invention described herein. Such equivalents are intended to be encompassed by the following claims.

What is claimed is:

1. A method of treating cancer in a subject in need thereof, the method comprising:
administering to the subject a therapeutically effective amount of an Enhancer of Zeste Homolog 2 (EZH2) inhibitor and a poly (ADP-ribose) polymerase 1 inhibitor.

2. The method of claim 1, wherein the cancer in the subject is characterized by elevated levels of expression of one or more of Cell Division Cycle-Associated Protein 3, CDC28 Protein Kinase Regulatory Subunit 2, MutY DNA Glycosylase, DNA Polymerase Epsilon 3, and Transforming Acidic Coiled-Coil Containing Protein 3 as compared to normal cells.

3. The method of claim 1, wherein the cancer is prostate cancer.

4. The method of claim 1, wherein the poly (ADP-ribose) polymerase 1 inhibitor is one or more of olaparib, rucaparib, niraparib (MK4827), talazoparib (BMN673), veliparib (ABT-888), iniparib, pamiparib, 3-Aminobenzamide (INO-1001), E7016 (GPI21016), CEP-8963, and CEP-9722.

5. The method of claim 1, wherein the poly (ADP-ribose) polymerase 1 inhibitor is olaparib.

6. The method of claim 1, wherein the EZH2 inhibitor is one or more of tazemetostat, 3-deazaneplanocin A (DZNep), EPZ005687, EI1, GSK126, EPZ-6438, GSK343, GSK503, CPI-1205, Constellation Compound 3, +OR-52, and +UNC1999.

7. The method of claim 1, wherein the EZH2 inhibitor is tazemetostat.

8. The method of claim 1, wherein the EZH2 inhibitor is 1-[(2S)-Butan-2-yl]-N-[(4,6-dimethyl-2-oxo-1H-pyridin-3-yl)methyl]-3-methyl-6-(6-piperazin-1-ylpyridin-3-yl)indole-4-carboxamide.

9. A method of evaluating response of a patient to an EZH2 inhibitor, the method comprising:
comparing expression levels of a plurality of biomarker genes in a first biological specimen from a patient to expression levels of the plurality of biomarker genes in a second biological specimen from the patient, wherein the second biological specimen is obtained by exposing the first biological specimen to an EZH2 inhibitor and a change in the expression levels of the plurality of biomarker genes is interpreted as the patient being responsive to the EZH2 inhibitor.

10. The method of claim 9, wherein the first biological specimen comprises prostate cancer cells.

11. The method of claim 9, wherein the plurality of biomarker genes includes three or more of Cell Division Cycle-Associated Protein 3, CDC28 Protein Kinase Regulatory Subunit 2, MutY DNA Glycosylase, DNA Polymerase Epsilon 3, and Transforming Acidic Coiled-Coil Containing Protein 3.

12. A method of evaluating response of a patient to an EZH2 inhibitor, the method comprising:
obtaining a first set of expression levels of a plurality of biomarker genes in a biological specimen from a patient and a second set of expression levels of the plurality of biomarker genes from normal cells, wherein the plurality of biomarker genes includes three or more of Cell Division Cycle-Associated Protein 3, CDC28 Protein Kinase Regulatory Subunit 2, MutY DNA Glycosylase, DNA Polymerase Epsilon 3, and Transforming Acidic Coiled-Coil Containing Protein 3; and
determining the patient to be responsive to an EZH2 inhibitor when the first set of expression levels of the plurality of biomarker genes in the biological specimen from the patient is elevated in comparison to the second set of expression levels of the plurality of biomarker genes from normal cells.

13. The method of claim 12, wherein the biological specimen comprises prostate cancer cells.

14. (canceled)

15. (canceled)


SEQUENCE LISTING (70 sequences)

SEQ ID NO: 1 - DNA, 20 bp, Artificial Sequence
  Description of Artificial Sequence Synthetic primer
ctaggagggtggaggtaggg

SEQ ID NO: 2 - DNA, 20 bp, Artificial Sequence
  Description of Artificial Sequence Synthetic primer
gccccaaacaggagtaatga

SEQ ID NO: 3 - DNA, 20 bp, Artificial Sequence
  Description of Artificial Sequence Synthetic primer
gccaggctcctgttttaatg

SEQ ID NO: 4 - DNA, 20 bp, Artificial Sequence
  Description of Artificial Sequence Synthetic primer
gcagtctccggttttgagag

SEQ ID NO: 5 - DNA, 20 bp, Artificial Sequence
  Description of Artificial Sequence Synthetic primer
tccaaccgaaactccaaaac

SEQ ID NO: 6 - DNA, 20 bp, Artificial Sequence
  Description of Artificial Sequence Synthetic primer
cttttcacccttcacggaaa

SEQ ID NO: 7 - DNA, 20 bp, Artificial Sequence
  Description of Artificial Sequence Synthetic primer
cctgctctgagtctgcactg

SEQ ID NO: 8 - DNA, 20 bp, Artificial Sequence
  Description of Artificial Sequence Synthetic primer
tcgaatctctcccatggttc

SEQ ID NO: 9 - DNA, 20 bp, Artificial Sequence
  Description of Artificial Sequence Synthetic primer
aggggaaggagagagcagtc

SEQ ID NO: 10 - DNA, 20 bp, Artificial Sequence
  Description of Artificial Sequence Synthetic primer
gggtgtttggtgtcataggg

SEQ ID NO: 11 - DNA, 20 bp, Artificial Sequence
  Description of Artificial Sequence Synthetic primer
tccttccatttccactcctg

SEQ ID NO: 12 - DNA, 20 bp, Artificial Sequence
  Description of Artificial Sequence Synthetic primer
tcttgctacaatggccttcc

SEQ ID NO: 13 - DNA, 20 bp, Artificial Sequence
  Description of Artificial Sequence Synthetic primer
gcgatttcttggcgttactt

SEQ ID NO: 14 - DNA, 19 bp, Artificial Sequence
  Description of Artificial Sequence Synthetic primer
tcggggaggttagagtgct

SEQ ID NO: 15 - DNA, 20 bp, Artificial Sequence
  Description of Artificial Sequence Synthetic primer
caatctcaggcgctctttgt

SEQ ID NO: 16 - DNA, 21 bp, Artificial Sequence
  Description of Artificial Sequence Synthetic primer
gagcgggaagaggaaagagta

SEQ ID NO: 17 - DNA, 20 bp, Artificial Sequence
  Description of Artificial Sequence Synthetic primer
tctccagcctatgcactcct

SEQ ID NO: 18 - DNA, 20 bp, Artificial Sequence
  Description of Artificial Sequence Synthetic primer
cttccatttgcacaagcaga

SEQ ID NO: 19 - DNA, 20 bp, Artificial Sequence
  Description of Artificial Sequence Synthetic primer
ccctctcttgccttcttcct

SEQ ID NO: 20 - DNA, 20 bp, Artificial Sequence
  Description of Artificial Sequence Synthetic primer
cgtcaggttttgggaacact

SEQ ID NO: 21 - DNA, 20 bp, Artificial Sequence
  Description of Artificial Sequence Synthetic primer
ccctaactgggaaaacacga

SEQ ID NO: 22 - DNA, 20 bp, Artificial Sequence
  Description of Artificial Sequence Synthetic primer
cgcccaggtaaatccaagta

SEQ ID NO: 23 - DNA, 20 bp, Artificial Sequence
  Description of Artificial Sequence Synthetic primer
aatagttcgggtgcgttttg

SEQ ID NO: 24 - DNA, 20 bp, Artificial Sequence
  Description of Artificial Sequence Synthetic primer
tcactggcccaatctttttc

SEQ ID NO: 25 - DNA, 20 bp, Artificial Sequence
  Description of Artificial Sequence Synthetic primer
ggggtggggaagtaatagga

SEQ ID NO: 26 - DNA, 20 bp, Artificial Sequence
  Description of Artificial Sequence Synthetic primer
ccttagcaggtgccatgagt

SEQ ID NO: 27 - DNA, 20 bp, Artificial Sequence
  Description of Artificial Sequence Synthetic primer
catggaccgtagtcttttct

SEQ ID NO: 28 - DNA, 20 bp, Artificial Sequence
  Description of Artificial Sequence Synthetic primer
cactgccaagaacgcactta

SEQ ID NO: 29 - DNA, 23 bp, Artificial Sequence
  Description of Artificial Sequence Synthetic primer
gcacaggattctaagtcctagca

SEQ ID NO: 30 - DNA, 20 bp, Artificial Sequence
  Description of Artificial Sequence Synthetic primer
ccagaccgggtgtgagtttt

SEQ ID NO: 31 - DNA, 21 bp, Artificial Sequence
  Description of Artificial Sequence Synthetic primer
atggtgcggactaaagcagac

SEQ ID NO: 32 - DNA, 23 bp, Artificial Sequence
  Description of Artificial Sequence Synthetic primer
cctcgatgaaactgatgtcgaat

SEQ ID NO: 33 - DNA, 21 bp, Artificial Sequence
  Description of Artificial Sequence Synthetic primer
aggaccaccgcatctctacat

SEQ ID NO: 34 - DNA, 21 bp, Artificial Sequence
  Description of Artificial Sequence Synthetic primer
aagtctggctcgttctcagtg

SEQ ID NO: 35 - DNA, 21 bp, Artificial Sequence
  Description of Artificial Sequence Synthetic primer
ctggagggtcttaaacatgcc

SEQ ID NO: 36 - DNA, 21 bp, Artificial Sequence
  Description of Artificial Sequence Synthetic primer
cactgctggtcttcataggtg

SEQ ID NO: 37 - DNA, 21 bp, Artificial Sequence
  Description of Artificial Sequence Synthetic primer
ccaaggcacatatccactgct

SEQ ID NO: 38 - DNA, 22 bp, Artificial Sequence
  Description of Artificial Sequence Synthetic primer
gtctcgatcacgtagaagacct

SEQ ID NO: 39 - DNA, 21 bp, Artificial Sequence
  Description of Artificial Sequence Synthetic primer
agtccactgagtaccggagac

SEQ ID NO: 40 - DNA, 21 bp, Artificial Sequence
  Description of Artificial Sequence Synthetic primer
catttcacgcatctggcgttc

SEQ ID NO: 41 - DNA, 21 bp, Artificial Sequence
  Description of Artificial Sequence Synthetic primer
atcagggtttgatgttcggct

SEQ ID NO: 42 - DNA, 19 bp, Artificial Sequence
  Description of Artificial Sequence Synthetic primer
gcgttgagcttgtgagcaa

SEQ ID NO: 43 - DNA, 23 bp, Artificial Sequence
  Description of Artificial Sequence Synthetic primer
ggctggagtctactatgtcttct

SEQ ID NO: 44 - DNA, 19 bp, Artificial Sequence
  Description of Artificial Sequence Synthetic primer
acctcggtgaagggagtcc

SEQ ID NO: 45 - DNA, 22 bp, Artificial Sequence
  Description of Artificial Sequence Synthetic primer
ttcgacgaacactacgagtacc

SEQ ID NO: 46 - DNA, 20 bp, Artificial Sequence
  Description of Artificial Sequence Synthetic primer
ggacaccaagtctcctccac

SEQ ID NO: 47 - DNA, 21 bp, Artificial Sequence
  Description of Artificial Sequence Synthetic primer
gtttcttacggcataggcgat

SEQ ID NO: 48 - DNA, 22 bp, Artificial Sequence
  Description of Artificial Sequence Synthetic primer
cacaaacgagtcaaattcagcc

SEQ ID NO: 49 - DNA, 21 bp, Artificial Sequence
  Description of Artificial Sequence Synthetic primer
ttcctcagtttcggcactcaa

SEQ ID NO: 50 - DNA, 22 bp, Artificial Sequence
  Description of Artificial Sequence Synthetic primer
ctcaaaaccaaaccgcaaatcc

SEQ ID NO: 51 - DNA, 19 bp, Artificial Sequence
  Description of Artificial Sequence Synthetic primer
gctgtacgccacatcctgt

SEQ ID NO: 52 - DNA, 21 bp, Artificial Sequence
  Description of Artificial Sequence Synthetic primer
ttcaatggggtaacgaaccgc

SEQ ID NO: 53 - DNA, 19 bp, Artificial Sequence
  Description of Artificial Sequence Synthetic primer
tgccacgtacagcagagac

SEQ ID NO: 54 - DNA, 21 bp, Artificial Sequence
  Description of Artificial Sequence Synthetic primer
caaaggcgatagaggcaatgg

SEQ ID NO: 55 - DNA, 21 bp, Artificial Sequence
  Description of Artificial Sequence Synthetic primer
tgttcgagtcacagaggatcg

SEQ ID NO: 56 - DNA, 21 bp, Artificial Sequence
  Description of Artificial Sequence Synthetic primer
acagtcgtagacgaaagctcc

SEQ ID NO: 57 - DNA, 22 bp, Artificial Sequence
  Description of Artificial Sequence Synthetic primer
gcaacggaatttcctgagatcc

SEQ ID NO: 58 - DNA, 19 bp, Artificial Sequence
  Description of Artificial Sequence Synthetic primer
acggagctatgttccccga

SEQ ID NO: 59 - DNA, 20 bp, Artificial Sequence
  Description of Artificial Sequence Synthetic primer
gtgccaactgtcacaccaca

SEQ ID NO: 60 - DNA, 21 bp, Artificial Sequence
  Description of Artificial Sequence Synthetic primer
gagtccacaagcattgcacac

SEQ ID NO: 61 - DNA, 21 bp, Artificial Sequence
  Description of Artificial Sequence Synthetic primer
ggaagacaaacctacagtcgc

SEQ ID NO: 62 - DNA, 23 bp, Artificial Sequence
  Description of Artificial Sequence Synthetic primer
tcctggtcagataggggttaaaa

SEQ ID NO: 63 - DNA, 20 bp, Artificial Sequence
  Description of Artificial Sequence Synthetic primer
agcaccacaagccacttcag

SEQ ID NO: 64 - DNA, 20 bp, Artificial Sequence
  Description of Artificial Sequence Synthetic primer
gggaaggagcggtcaaactg

SEQ ID NO: 65 - DNA, 23 bp, Artificial Sequence
  Description of Artificial Sequence Synthetic primer
agaggcttccgatttcgatatgg

SEQ ID NO: 66 - DNA, 21 bp, Artificial Sequence
  Description of Artificial Sequence Synthetic primer
ggataggtctttcggcccttc

SEQ ID NO: 67 - DNA, 21 bp, Artificial Sequence
  Description of Artificial Sequence Synthetic primer
agtgctcctatatccctccct

SEQ ID NO: 68 - DNA, 22 bp, Artificial Sequence
  Description of Artificial Sequence Synthetic primer
gctgggaaccaatctcatcttt

SEQ ID NO: 69 - DNA, 20 bp, Artificial Sequence
  Description of Artificial Sequence Synthetic primer
cgagatccctccaaaatcaa

SEQ ID NO: 70 - DNA, 20 bp, Artificial Sequence
  Description of Artificial Sequence Synthetic primer
ttcacacccatgacgaacat